(19) United States
(12) Patent Application Publication
(10) Pub. No.: US 2003/0235631 A1
(43) Pub. Date: Dec. 25, 2003

(54) Combination treatment for depression and anxiety

(75) Inventors: Susan B. Sobolov-Jaynes, Ivoryton, CT (US); Christopher J. Schmidt, Old Lyme, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/387,060

(22) Filed: Mar. 12, 2003

Related U.S. Application Data
(60) Provisional application No. 60/389,181, filed on Jun. 17, 2002.

(51) Int. Cl. A61K 35/78; A61K 31/551; A61K 31/5513; A61K 31/537; A61K 31/496; A61K 31/495; A61K 31/405; A61K 31/137

(52) U.S. Cl. 424/730; 514/231.2; 514/253.04; 514/255.04; 514/220; 514/221; 514/419; 514/649; 514/651; 514/540; 514/254.11

(57) ABSTRACT

The present invention relates to a method of treating depression or anxiety in a mammal, including a human, by administering to the mammal a PDE IV inhibitor in combination with an antidepressant or an anxiolytic agent. It also relates to pharmaceutical compositions containing a pharmaceutically acceptable carrier, a PDE IV inhibitor and an anxiolytic agent or antidepressant.

### BACKGROUND OF THE INVENTION

The present invention relates to a method of treating depression or anxiety in a mammal, including a human, by administering to the mammal a PDE IV inhibitor in combination with an antidepressant or an anxiolytic agent. It also relates to pharmaceutical compositions containing a pharmaceutically acceptable carrier, a PDE IV inhibitor and an anxiolytic agent or antidepressant.

Major depression is characterized by feelings of intense sadness and despair, mental slowing and loss of concentration, pessimistic worry, agitation, and self-deprecation. Physical changes also occur, especially in severe or “melancholic” depression. These include insomnia or hypersomnia, anorexia and weight loss (or sometimes overeating), decreased energy and libido, and disruption of normal circadian rhythms of activity, body temperature, and many endocrine functions.

Treatment regimens commonly include the use of tricyclic antidepressants, monoamine oxidase inhibitors, some psychotropic drugs, lithium carbonate, and electroconvulsive therapy (ECT) (see R. J. Baldessarini in Goodman & Gilman's The Pharmacological Basis of Therapeutics, 9th Edition, Chapter 19, McGraw-Hill, 1996 for a review). More recently, new classes of antidepressant drugs are being developed including selective serotonin reuptake inhibitors (SSRIs), specific monoamine reuptake inhibitors and 5-HT_{IA} receptor agonists, antagonists and partial agonists.

Anxiety is an emotional condition characterized by feelings such as apprehension and fear accompanied by physical symptoms such as tachycardia, increased respiration, sweating and tremor. It is a normal emotion but when it is severe and disabling it becomes pathological.

Anxiety disorders are generally treated using benzodiazepine sedative-antianxiety agents. Potent benzodiazepines are effective in panic disorder as well as in generalized anxiety disorder, however, the risks associated with drug dependency may limit their long-term use. 5-HT_{IA} receptor partial agonists also have useful anxiolytic and other psychotropic activity, and less likelihood of sedation and dependence (see R. J. Baldessarini in Goodman & Gilman's Tite Pharmacological Basis of Therapeutics, 9th Edition, Chapter 18, McGraw-Hill, 1996 for a review).

### SUMMARY OF THE INVENTION

The present invention relates to a pharmaceutical composition for the treatment of anxiety or depression comprising: (a) a compound that exhibits activity as an antianxiety (i.e., anxiolytic) agent or an antidepressant, or a pharmaceutically acceptable salt thereof; (b) a PDE IV inhibitor or pharmaceutically acceptable salt thereof; and (c) a pharmaceutically acceptable carrier; wherein the active agents “a” and “b” above are present in amounts that render the composition effective in treating, respectively, anxiety or depression.

This invention also relates to a method of treating anxiety or depression in a mammal, comprising administering to said mammal, respectively, an anxiolytic or antidepressant effective amount of a pharmaceutical composition comprising: (a) a compound that exhibits activity as, respectively, an anxiolytic agent or an antidepressant, or a pharmaceutically acceptable salt thereof; (b) a PDE IV inhibitor or pharmaceutically acceptable salt thereof; and (c) a pharmaceutically acceptable carrier; wherein the active agents “a” and “b” above are present in amounts that render the composition effective in treating, respectively, anxiety or depression.

This invention also relates to a method of treating anxiety or depression in a mammal, comprising administering to said mammal: (a) a compound that exhibits activity as, respectively, an anxiolytic agent or an antidepressant, or a pharmaceutically acceptable salt thereof; and (b) a PDE IV inhibitor or pharmaceutically acceptable salt thereof; wherein the active agents “a” and “b” above are present in amounts that render the combination of the two agents effective in treating, respectively, anxiety or depression.

It will be appreciated that when using a combination method of the present invention, referred to immediately above, both the PDE IV inhibitor and the antidepressant or anti-anxiety agent will be administered to a patient within a reasonable period of time. The compounds may be in the same pharmaceutically acceptable carrier and therefore administered simultaneously. They may be in separate pharmaceutical carriers such as conventional oral dosage forms that are taken simultaneously. The term combination, as used above, also refers to the case where the compounds are provided in separate dosage forms and are administered sequentially. Therefore, by way of example, the antidepressant or anxiolytic agent may be administered as a tablet and then, within a reasonable period of time, the PDE IV inhibitor may be administered either as an oral dosage form such as a tablet or a fast-dissolving oral dosage form. By a “fast dissolving oral formulation” is meant, an oral delivery form which when placed on the tongue of a patient, dissolves within about seconds.

The compositions of the present invention that contain an PDE IV inhibitor and an antidepressant are useful for the treatment of depression. As used herein, the term “depression” includes depressive disorders, for example, single episodic or recurrent major depressive disorders, and dysthymic disorders, depressive neurosis, and neurotic depression; melancholic depression including anorexia, weight loss, insomnia and early morning waking, and psychomotor retardation; a typical depression (or reactive depression) including increased appetite, hypersomnia, psychomotor agitation or irritability, anxiety and phobias, seasonal affective disorder, or bipolar disorders or manic depression, for example, bipolar I disorder, bipolar II disorder and cyclothymic disorder.

Other mood disorders encompassed within the term “depression” include dysthymic disorder with early or late onset and with or without a typical features; dementia of the Alzheimer's type, with early or late onset, with depressed mood; vascular dementia with depressed mood, disorders induced by alcohol, amphetamines, cocaine, hallucinogens, inhalants, opioids, phencyclidine, sedatives, hypnotics, anxiolytics and other substances; schizoaffective disorder of the depressed type; and adjustment disorder with depressed mood.

The compositions of the present invention that contain a PDE IV inhibitor an anxiolytic agent are useful for the treatment of anxiety. As used herein, the term “anxiety” includes anxiety disorders, such as panic disorder with or without agoraphobia, agoraphobia without history of panic disorder, specific phobias, for example, specific animal phobias, social phobias, obsessive-compulsive disorder, stress disorders including post-traumatic stress disorder and acute stress disorder, and generalized anxiety disorders.

“Generalized anxiety” is typically defined as an extended period (e.g. at least six months) of excessive anxiety or worry with symptoms on most days of that period. The anxiety and worry is difficult to control and may be accompanied by restlessness, being easily fatigued, difficulty concentrating, irritability, muscle tension, and disturbed sleep.

“Panic disorder” is defined as the presence of recurrent panic attacks followed by at least one month of persistent concern about having another panic attack. A “panic attack” is a discrete period in which there is a sudden onset of intense apprehension, fearfulness or terror. During a panic attack, the individual may experience a variety of symptoms including palpitations, sweating, trembling, shortness of breath, chest pain, nausea and dizziness. Panic disorder may occur with or without agoraphobia.

“Phobias” includes agoraphobia, specific phobias and social phobias. “Agoraphobia” is characterized by an anxiety about being in places or situations from which escape might be difficult or embarrassing or in which help may not be available in the event of a panic attack. Agoraphobia may occur without history of a panic attack. A “specific phobia” is characterized by clinically significant anxiety provoked by feared object or situation. Specific phobias include the following subtypes: animal type, cued by animals or insects; natural environment type, cued by objects in the natural environment, for example storms, heights or water; blood-injection-injury type, cued by the sight of blood or an injury or by seeing or receiving an injection or other invasive medical procedure; situational type, cued by a specific situation such as public transportation, tunnels, bridges, elevators, flying, driving or enclosed spaces; and other type where fear is cued by other stimuli. Specific phobias may also be referred to as simple phobias. A “social phobia” is characterized by clinically significant anxiety provoked by exposure to certain types of social or performance circumstances. Social phobia may also be referred to as social anxiety disorder.

Other anxiety disorders encompassed within the term “anxiety” include anxiety disorders induced by alcohol, amphetamines, caffeine, cannabis, cocaine, hallucinogens, inhalants, phencychdine, sedatives, hypnotics, anxiolytics and other substances, and adjustment disorders with anxiety or with mixed anxiety and depression.

Anxiety may be present with or without other disorders such as depression in mixed anxiety and depressive disorders. The compositions of the present invention are therefore useful in the treatment of anxiety with or without accompanying depression.

The compositions of the present invention are especially useful for the treatment of depression or anxiety where the use of an antidepressant or anxiolytic agent, respectively, is generally prescribed. By the use of a combination of a PDE IV inhibitor and an antidepressant or anxiolytic agent in accordance with the present invention, it is possible to treat depression and/or anxiety in patients for whom conventional antidepressant or antianxiety therapy might not be wholly successful or where dependence upon the antidepressant or antianxiety therapy is prevalent.

Suitable classes of antidepressant agents that may be used in the present invention include norepinephrine reuptake inhibitors, selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists and a typical antidepressants.

Another class of antidepressant agents that may be used in the present invention are noradrenergic and specific serotonergic antidepressants (NaSSAs). A suitable example of a NaSSA is mirtazapine.

Suitable norepinephrine reuptake inhibitors that may be used in the present Invention include tertiary amine tricyclics and secondary amine tricyclics. Suitable examples of tertiary amine tricyclics include: amitriptyline, clomipramine, doxepin, imipramine and trimipramine, and pharmaceutically acceptable salts thereof. Suitable examples of secondary amine tricyclics include: amoxapine, desipramine, maprotiline, nortriptyline and protriptyline, and pharmaceutically acceptable salts thereof.

Another norepinephrine reuptake inhibitor that may be used in the present invention is reboxetine.

Suitable selective serotonin reuptake inhibitors that may be used in the present invention include: fluoxetine, fluvoxamine, paroxetine and sertraline, and pharmaceutically acceptable salts thereof.

Suitable monoamine oxidase inhibitors that may be used in the present invention include: isocarboxazid, pheneizine, tranylcypromine and selegiline, and pharmaceutically acceptable salts thereof.

Suitable reversible inhibitors of monoamine oxidase that may be used in the present invention include: moclobemide, and pharmaceutically acceptable salts thereof.

Suitable serotonin and noradrenahne reuptake inhibitors that may be used in the present invention include: venlafaxine, and pharmaceutically acceptable salts thereof.

Suitable CRF antagonists that may be used in the present invention include those compounds described in International Patent Specification Nos. WO 94/13643, WO 94/13644, WO 94/13661, WO 94/13676 and WO 94/13677.

Suitable a typical antidepressants that may be used in the present invention include: bupropion, lithium, nefazodone, trazodone and viloxazine, and pharmaceutically acceptable salts thereof Another suitable a typical antidepressant is sibutramine.

Other antidepressants that may be used in the present invention include adinazolam, alaproclate, amineptine, amitriptyline/chlordiazepoxide combination, atipamezole, azamianserin, bazinaprine, befuraline, bifemelane, binodaline, bipenamol, brofaromine bupropion, caroxazone, cericlamine, cianopramine, cimoxatone, citalopram, clemeprol, clovoxamine, dazepinil, deanol, demexiptiline, dibenzepin, dothiepin, droxidopa, enefexine, estazolam, etoperidone, femoxetine, fengabine, fezolamine, fluotracen, idazoxan, indalpine, indeloxazine, iprindole, levoprotiline, litoxetine, lofepramine, medifoxamine, metapramine, metralindole, mianserin, milnacipran, minaprine, mirtazapine, montirelin, nebracetam, nefopam, nialamide, nomifensine, norfluoxetine, orotirelin, oxaflozane, pinazepam, pirlindone, pizotyline, ritanserin, rolipram, sercloremine, setiptiline, sibutramine, sulbutiamine, sulpiride, teniloxazine, thozalinone, thymoliberin, tianeptine, tiflucarbine, tofenacin, tofisopam, toloxatone, tomoxetine, veralipride, viqualine, zimelidine and zometrapine, and pharmaceutically acceptable salts thereof, and St. John's wort herb, or Hypericuin perforatum, or extracts thereof.

Suitable classes of anti-anxiety agent that may be used in the present invention include benzodiazepines and 5-HT_{IA} agonists or antagonists, especially 5-HT_{IA} partial agonists, and corticotropin releasing factor (CRF) antagonists. In addition to benzodiazepines, other suitable classes of antianxiety agent are nonbenzodiazepine sedative-hypnotic drugs such as zolpidem; mood-stabilizing drugs such as clobazam, gabapentin, lamotrigine, loreclezole, oxcarbamazepine, stiripentol and vigabatrin; and barbiturates.

Suitable benzodiazepines that may be used in the present invention include: alprazolam, chlordiazepoxide, clonazepam, chlorazepate, diazepam, halazepam, lorazepam, oxazepam and prazepam, and pharmaceutically acceptable salts thereof.

Suitable 5-HT_{IA} receptor agonists or antagonists that may be used in the present invention include, in particular, the 5-HT_{IA} receptor partial agonists buspirone, flesinoxan, gepirone and ipsapirone, and pharmaceutically acceptable salts thereof. An example of a compound with 5-HT_{IA} receptor antagonist/partial agonist activity is pindolol.

Suitable CRF antagonists that may be used in the present invention include those compounds described in International Patent Application Nos. WO 94/13643, WO 94/13644, WO 94/13661, WO 94/13676 and WO 94/13677.

Another class of anti-anxiety agent that may be used in the present invention are compound having muscarinic cholinergic activity. Suitable compounds in this class include m1 muscarinic cholinergic receptor agonists such as those compounds described in European Patent Application Nos. 0 709 093, 0 709 094 and 0 773 021, and International Patent Application No. WO 96/12711.

Another class of anti-anxiety agent that may be used in the present invention are compounds acting on ion channels. Suitable compounds in this class include carbamazepine, lamotrigine and valproate, and pharmaceutically acceptable salts thereof.

Specific examples of anti-depressants and anti-anxiety agents that may be used in the methods and pharmaceutical compositions of this invention are the following compounds

citalopram; Disclosed in EP 347,066 on Dec. 20, 1989.

fluoxetine; Disclosed in U.S. Pat. No. 4,018,895 on Apr. 19, 1977.

sertraline; Disclosed in U.S. Pat. No. 4,536,518 on Aug. 20, 1985.

paroxetine; Disclosed in WO 97/24323 on Jul. 10, 1997

nefazadone; Disclosed in Neuropharmacology (1986) 25 (127, 1301-1306).

bupropion; Disclosed in U.S. Pat. No. 3,885,046 on Jun. 20, 1975.

escitalopram; Disclosed in EP 347,066 on Dec. 20, 1989.

zimelidine; Disclosed in EP 303,961 on Feb. 22, 1989.

fluvoxamine; Disclosed in WO 96/41633 on Dec. 27, 1996.

duloxetine; Disclosed in EP 457,559 on Nov. 21, 1991.

milnacipran; Disclosed in FR 2,581,060 on Oct. 31, 1986.

venlafaxine; Disclosed in EP 112,669 on Jul. 4, 1984.

trazodone; Disclosed in U.S. Pat. No. 3,381,009 on Apr. 30, 1968.

mirtazapine; Disclosed in GB 1,543,171 on Mar. 28, 1979.

amitriptyline; Disclosed in BE 634,372 on Jan. 2, 1964.

imipramine; Disclosed in FR 5218 on Aug. 7, 1967.

lubazodone hydrochloride; Disclosed in WO 94/18182 on Feb. 8, 1994.

[Morpholine, 2-[[(7-fluoro-2,3-dihydro-1H-inden-4yl)oxy]methyl]-, hydrochloride, (2S)-(9Cl];

2-Benzofuran-carboxamide, 5-[4-[4-(5-cyano-1H-indol-3-yl)butyl]-1-piperazinyl]-(9Cl);

mianserin; Disclosed in DE 2,505,239 on Aug. 14, 1975.

tianeptine; Disclosed in JP 53,005,661 on Mar. 1, 1978.

minaprine; Disclosed in GB 1,345,880 on Feb. 6, 1974.

pheneizine (MAO-I); Disclosed in U.S. Pat. No. 3,334,017 on Aug. 1, 1967

isocarboxazid (MAO-I); Disclosed in EP 563,507 on Oct. 6, 1993.

tranylcypromine (MAO-I) Disclosed in U.S. Pat. No. 4,016,204 on Apr. 5, 1997 and

St John's Wort; Disclosed in WO 99/66914 on Dec. 29, 1999.

The PDE IV inhibitors are selected from the group consisting of and their pharmaceutically acceptable salts:

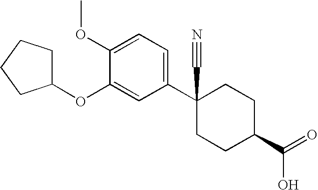

### 1. Cilomilast Claimed in EP 1188438 Issued Mar. 20, 2002

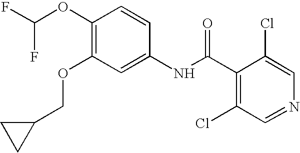

### 2. Roflumilast claimed in U.S. Pat. No. 5,958,926

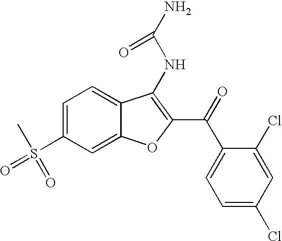

### 3. BAY 19-8004 [2-(2,4-Dichloro-benzoyl-6-methanesulfonyl-benzofuran-3-yl]-urea Published in WO115677 on Mar. 8, 2001.

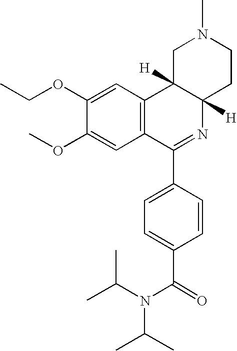

### 4. Pumafentrine Published in WO9821208 on May 22, 1998.

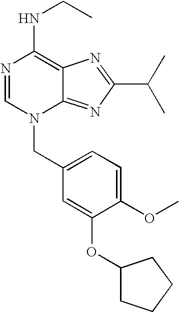

### 5. V-11294A 3H-Purin-6-amine 3-[(3-cyclopentyloxy-)-4-methoxy-phenyl]methyl]-N-ethyl-8-(1-methylethyl-, Monohydrochloride Published in WO 0059449 on Oct. 12, 2000.

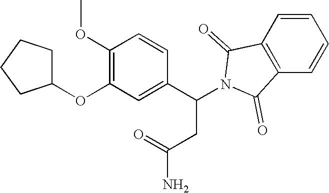

### 6. CDC-801 2H-I soindole-2-propan-amide B-[3-cyclopentoxy)-4 methoxyphenyl]-1,3-dihydro-1,3-cloxo-(9Cl) claimed in U.S. Pat. No. 5,728,844 issued Mar. 17, 1998

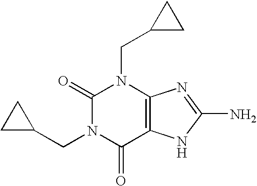

### 7. Cipamfylline Published in WO 9920625 on Apr. 29, 1999

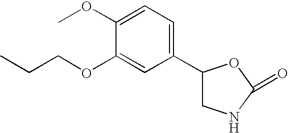

### 8. Mesopram claimed in German Patent No. 19540475 issued Apr. 24, 1997

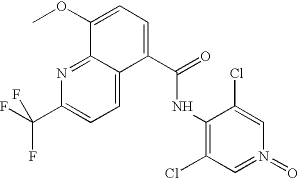

### 9. SCH-351591-5-Quinolinecarboxamide, N-(3,5-d ichloro-1-oxido-4-pyridinyl)-8-methoxy-2-(trifluorometomethyl)-(9Cl) Published in WO 0026208 May 11, 2000

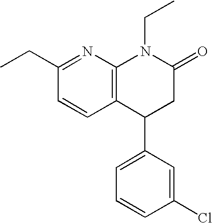

### 10. YM-976 Pyrido[2,3-d]pyrimidin-2(1H)-one, 4-(3-chloro-phenyl)-1,7-diefyl-(9Cl) Published in WO 9719078 on May 29, 1997

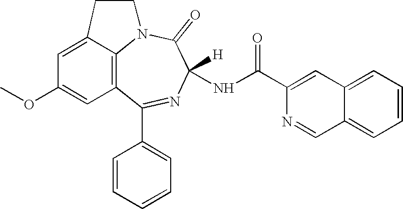

### 11. Cl-1044 3-pyridine carboxamide, N-(9-amino-3,4,6,7-tetrahydro-4-oxo-1-phenyl pyrrolo[3,2,1-jk] [1,4]benzodiazepin-3-yl)(R)-(9Cl) Published in WO 9736905 on Oct. 9, 1997

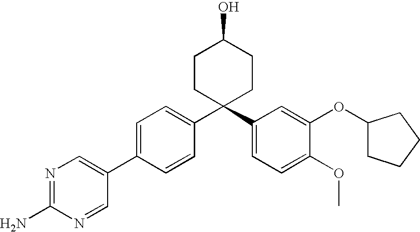

### 12. Cyclohexanol 4-[4-2-amino-5-pyrimidinyl)phenyl]-4-3-(cyclo-pentyloxy) -4-methoxylphenyl]-trans-(9Cl) Published in WO 110385A2 on Feb. 15, 2001

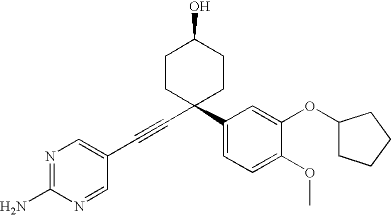

### 13. Cyclohexanol, 4-[(2-amino-5-pyrinidinyl, ethynly]-4-[3-(cyclopentoxy)-4-methoxypheny]-cis-(9Cl) Published in WO 9619988A1 on Jul. 4, 1996

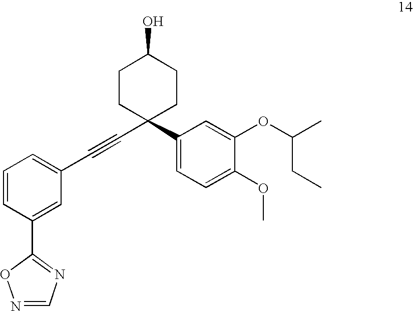

### 4-(3-sec-Butoxy-4-methoxy-phenyl-4-(3-[1,2,4] oxadiazol-5-yl-phenylethynyl)-cyclohexanol

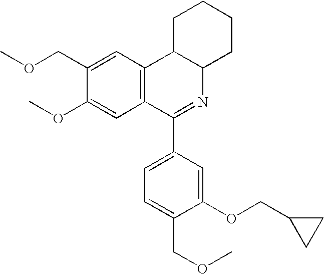

### 15. 6-(3-Cyclopropylmethoxy-4-methoxymethyl-phenyl-8-methoxy-9-methoxy-methyl -1,2,3,4,4a,10b-hexahydro-phenanthridine

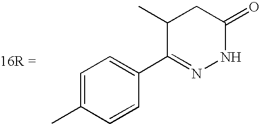

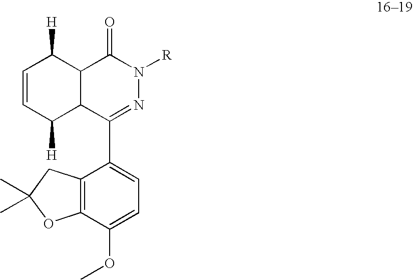

### 4-(7-Methoxy-2,2-dimethyl-2-3-dihydro-benzofuran-4-yl)-2-[4-(4-methyl-6-oxo-1,4,5,6-tetrahydro-pyridazin-3-yl)-phenyl]-4a,5,8,8a-tetrahydro-2H-phthalazin-1-one

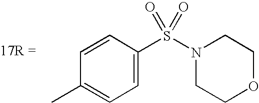

### Morpholine, 4-[[4-[(4aR,8aS)-4-(2,3-dihydro-7-methoxy-2,2-dimethyl-4-benzofuranyl)-4a,5,8,8a-tetrahydro-1-oxo-2(1H)-phthalazinyl]phenyl]sulfonyl]-,rel-(9Cl) Disclosed in WO 01360766 on May 3, 2001

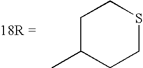

### 1(2H)-Phthalazinone, 4-(2,3-dihydro-7-methoxy-2,2-dimethyl-4-benzofuranyl)-4a,5,8,8a-tetrahydro-2-(tetrahydro-2H-thiopyran-4-yl)-, (4aR,8aS)-rel-(9Cl) Disclosed in WO1370777 On May 3, 2001

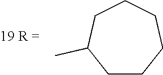

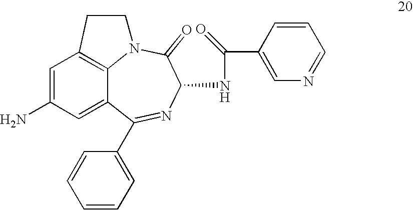

Disclosed in J. of Med. Chem, Volume 43 No. 25, pp. 4850-4867 published in 2000.

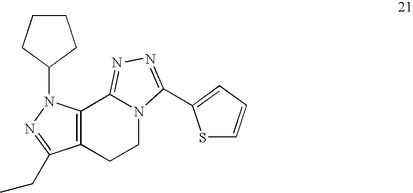

### 21 Tofimilast Disclosed in EP 2000-302947 on Apr. 7, 2000 5H-Pyrazolo[3,4-c]-1,2,4-triazolo[4,3-a]pyridine, 9-cyclopentyl-7-ethyl-6,9-dihydro-3-(2-thienyl)-(9Cl)

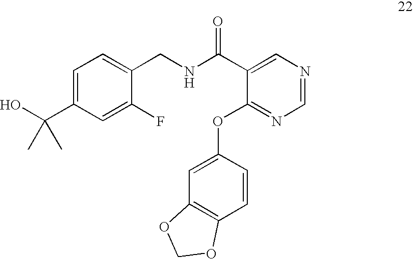

### 5-Pyrimidinecarboxamide, 4-(1,3-benzodioxol-5-yloxy)-N-[[2-fluoro-4(1-hydroxy-1-methylethyl)phenyl]methyl]-(9Cl) Disclosed in WO01 57025 On Aug. 19, 2001

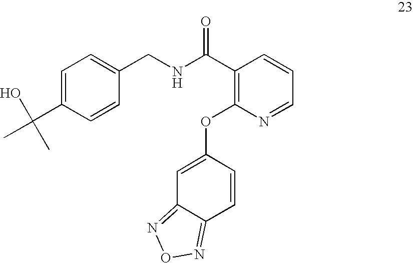

### 2-(Benzo[1,2,5]oxadiazol-5-yloxy)-N-[4-(1-hydroxy-1-methyl-ethyl)-benzyl]-nicotinamide

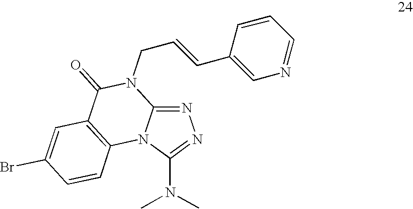

### 24

Disclosed in WO 0066584 on Nov. 9, 2001, [1,2,4]Triazolo[4,3-a]quinazolin-5(4H)-one,7-bromo-1-(dimethylamino)-4-[3-(3-pyridinyl)-2-propenyl]-(9Cl)

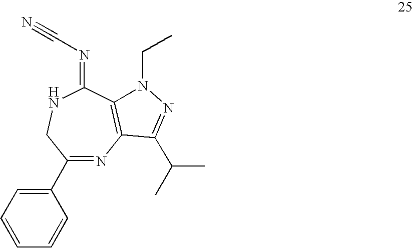

### 25 Cyanamide, [1-ethyl-1,6-dihydro-3-(1-methylethyl)-5-phenylpyrazolo[4,3-e][1,4]diazepin-8-yl]-(9Cl) Disclosed in WO0149689 On Jul. 12, 2001

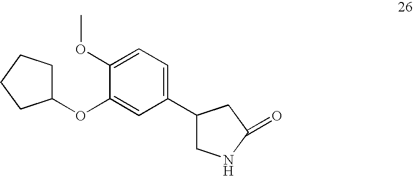

### 26 2-pyrrolidinone, 4-[3-cyclopentyloxy)-4-methoxyphenyl]-(9Cl) Disclosed in WO9202220 On Feb. 20, 1992

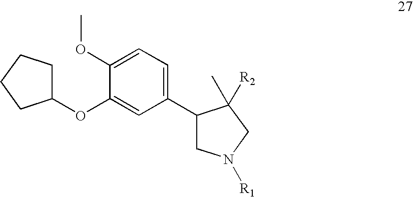

27A=R₁=CO₂CH₃,27B=benzyl R₂=various groups

### 27

27A. 1-Pyrrolidinecarboxylic acid, 4-[3-(cyclopentyloxy)-4-methoxyphenyl]3-formyl-3-methyl-,methyl ester, (3S,4S)-(9Cl)

27A was disclosed in WO0146136 on Jun. 28, 2001.

27B was disclosed in WO0147879 on Jul. 5, 2001.

27B. 3-Pyrrolidinemethanamine, 4-[[3-(cyclopentyloxy)-4-methoxyphenyl]-N,3-dimethyl-1-(phenylmethyl)-, (3R,4S)-(9Cl)

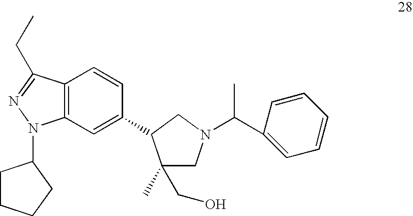

### 28 [4-(1-Cyclopentyl-3-ethyl-1H-indazol-6-yl)-3-methyl-1-(1-phenyl-ethyl)-pyrrolidin-3-yl]-methanol

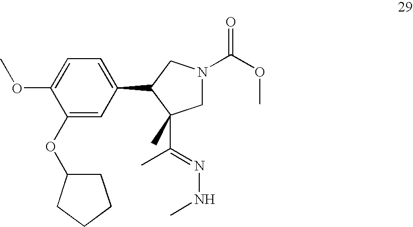

### 29 1-Pyrrolidinecarboxylic acid, 4-[3-(cyclopentyloxy)-4-methoxyphenyl]-3-methyl -3-[1-(methylhydrazono)ethyl]-,methyl ester (9Cl) Disclosed in WO 0146136 On Jun. 28, 2001

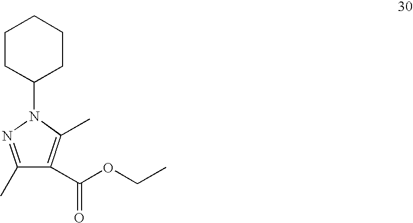

### 30 1H-Pyrazole-4-carboxylic acid, 1-cyclohexyl-3,5-dimethyl-,ethyl ester (9Cl) Disclosed in WO 146172 on Jun. 28, 2001

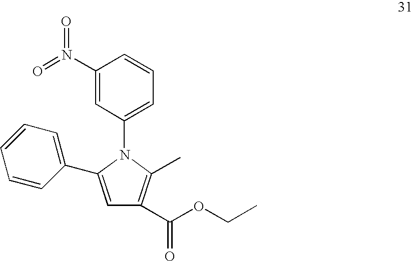

### 31 1H-Pyrrole-3-carboxylic acid, 2-methyl-1-(3-nitrophenyl)-5-phenyl-, ethyl ester (9Cl) Disclosed in WO 147880 On Jul. 5, 2001

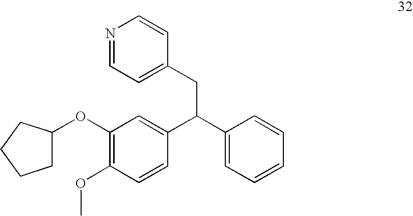

### 32 Pyridine, 4-[2-[3-(cyclopentyloxy)-4-methoxyphenyl]-2-phenylethyl]-(9Cl) Disclosed in WO9414742 On Jul. 7, 1994

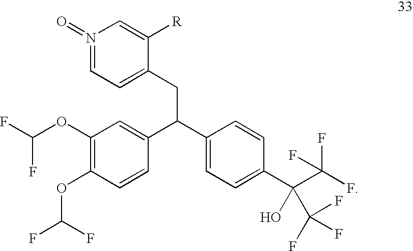

### 33. Benzenemethanol, 4-[1-[3,4-bis(difluormethoxy)phenyl]-2-(1-oxido-4-pyridinyl)ethyl]-α, α-bis(trifluoromethyl)-(9Cl) Disclosed in WO 9722586 on Jun. 26, 1997

### 34. R=CH₃ 2-{4-[1-(3,4-Bis-difluoromethoxy-phenyl)-2-(3-methyl-1-Oxy-pyridin-4-yl)-ethyl]-phenyl}-1,1,1,3,3,3-hexafluoro-proppane

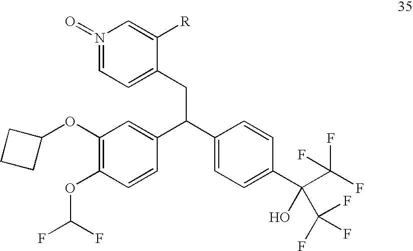

### 35 A. =H -2-{4-[1-(3-Cyclobutyloxy-4-difluoromethoxy-phenyl) -2(3-methyl-1-oxy-pyridin-4-yl) -ethyl]-phenyl}-1,1,1,3,3,3-hexfluoro-propan-2-ol

### B. =Me 2-{4-[1-(3-Cyclobutyloxy-4-difluoromethoxy-phenyl) B=Me -2-(1-oxy-pyridin-4-yl-ethyl]-phenyl}-1,1,1,3,3,3-hexafluoro-propan-2-ol

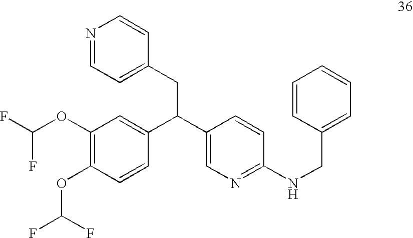

### 36 Disclosed in WO0068198 on Nov. 16, 2001 2-Pyridinamine, 5-[1-[3,4-bis(difluormethoxy)phenyl]-2(4-pyridinyl) ethyl]-N-(phenylmethyl)-(9Cl)

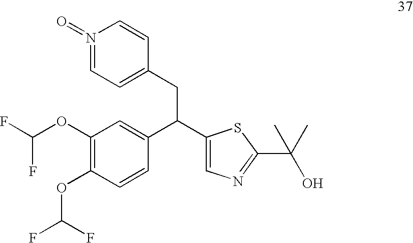

### 37 2-{5-[1-(3,4-Bis-difluoromethoxy-phenyl)-2-(1-oxy-pyridin-4-yl) -ethyl]-thiazol-2-yl}-propan-2-ol

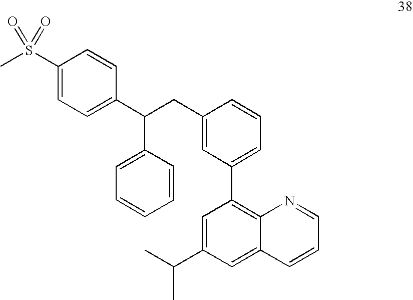

### 38 6-Isopropyl-8-{3-[2-(4-methanesulfonyl-phenyl)-2-phenyl-ethyl-phenyl}quinoline

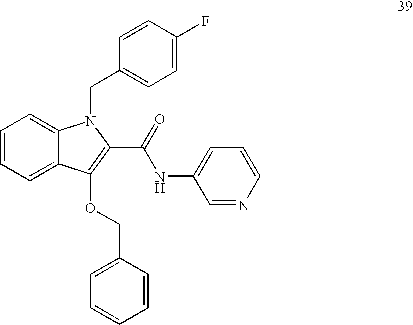

### 39 1H-Indole-2-carboxamide, 39 1-[(4-flurophenyl)methyl]-3-(phenylmethoxy)-N-3-pyridinyl-(9Cl) Disclosed in WO0164639 on Sep. 7, 2001

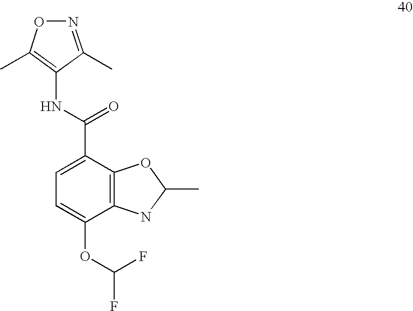

### 40 4-Difluoromethoxy-2-methyl-2,3-dihydro-benzooxazole-7-carboxylic Acid (3,5-dimethyl-isoxazol-4-yl)-amide

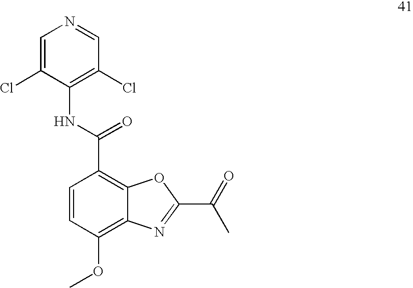

### 41 2-Acetyl-4-difluoromethoxy-benezooxazole-7-carboxylic Acid (3,5-dichloro-pyridin-4-yl-amide

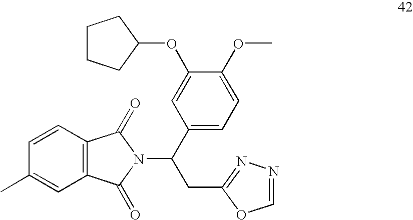

### 42 1H-Isoindole-1,3)2H)-dione, 2-[1-[3-(cyclopentyloxy)-4-methoxyphenyl]-2-(1,3,4-oxadiazol-2-yl) ethyl]-5-methyl-(9Cl) Disclosed in WO-0146183 on Jun. 28, 2001.

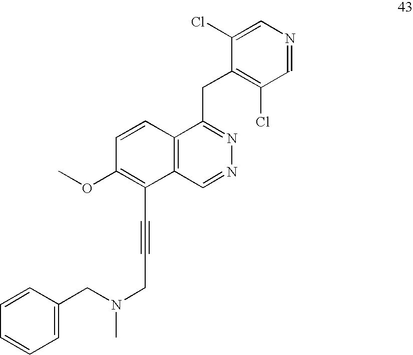

### 43 Benezenemethanamine, N-[3-]1-[(3,5-dichloro-4-pyridinyl)methyl]-6-methoxy-5-phthalazinyl] -2-propynyl]-N-methyl-(9Cl) Disclosed in WO 0005218 on Feb. 3, 2000.

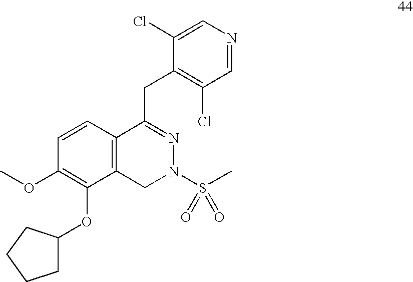

### 44 8-Cyclopentyloxy-4-(3,5-dichloro-pyrid in-4-yl methyl)-2-methanesulfonyl-7-methoxy-1,2-dihydro-phthalazine

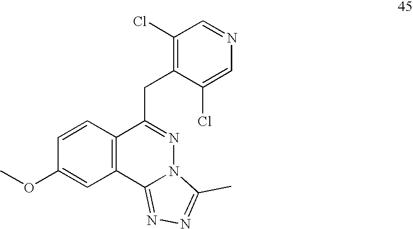

### 45 1,2,4-Triazole[3,4-a]phthalazine, 6-[3, 5-dichloro-4-pyridinyl)methyl]-9-methoxy-3-methyl-(9Cl) Disclosed in WO 0026218 on May 11, 2000.

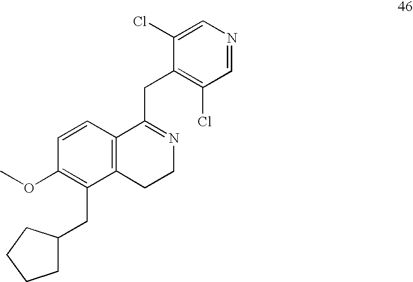

### 46 Isoquinoline, 5-(cyclopentylmethyl)-1-[(3,5-dicloro-4-pyridinyl)methyl]-3-4-dihydro-6-methoxy-(9Cl) Disclosed in WO0021947 on Apr. 20, 2000.

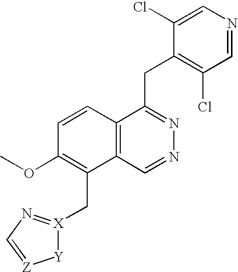

47a X=CH, Y=S, Z=CH 1-(3,5-Dichloro-pyridin-4-ylmethyl)-6-methoxy-5-thiazol-2-ylmethyl-phthalazine

47b X=N, Y=CH, Z=N 1-(3,5-Dichloro-pyridin-4-ylmethyl)-6-methoxy-5-(5H-[1,2,4]triazol-1-ylmethyl)-phthalazine

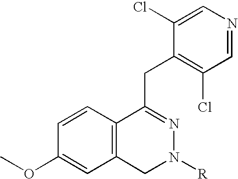

### 48 R=SO₂CH₃

### 49 R=COCH₂Ph Phthalazine, 4-[(3,5-dichloro-4-pyridinyl)methyl]-1,2-dihydro-7-methoxy-2-(phenlacetyl)-(9Cl) Both Disclosed in WO 0005218 on Feb. 3, 2000

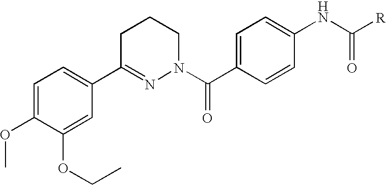

### 50 R=alkoxy {4-[3-(3-Ethoxy-4-methoxy-phenyl)-5,6-dihydro-4H-pyridazine-1-carbonyl}-carbamic Acid Methyl Ester

### 51 R=hetroaryl 4-Pyridinecarboxamide, N-[4-[[3-(3-ethoxy-4-methoxyphenyl)-5,6-dihydro-1(4H)-Pyridazinyl]carbonyl]phenyl-(9Cl).

Disclosed in WO 9806704 in Feb. 19, 1998

### 52 R=NH2, alkylNH 1-{4-[3-(3-Ethoxy-4-methoxy-phenyl)-5.6-dihydro-4H-pyridazine-1-carbonyl]-phenyl}-3-methyl-urea

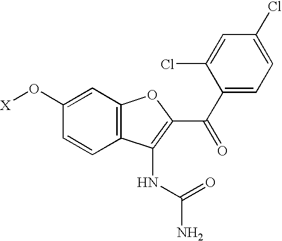

### 53 X=substittued alkyl—Disclosed in WO 0069844 on Nov. 23, 2000 Urea, [2-(2,4-dichlorobenzoyl)-6-[(3E)-3-pentenyloxy]-3-benzofuranyl]-(9Cl)

### 54 X=alkylsulfonly, arylsulfonyl Disclosed in WO0069844 on Nov 23,2000 Benzene sulfonic acid, 4-[(dimethylamino)sulfonyl]amino],-3-[(aminocarbonyl)amino]-2-(2,4,-dichlorobenzoyl)-6-benzofuranyl Ester (9Cl)

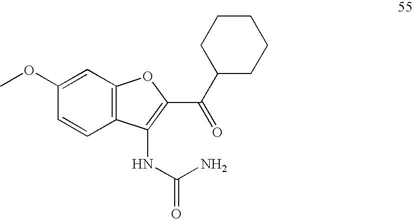

### 55 Urea, [2-(cyclohexylcarbonyl)-6-methoxy-3-benzofuranyl]-(9Cl) Disclosed in WO0069843 on Nov. 23, 2000

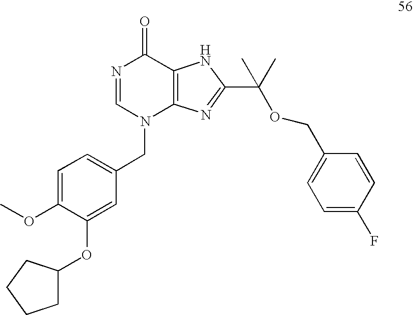

### 56

### 6H-Purin-6-one, 3-[[3-(cyclopentyloxy)-4methoxyphenyl]methyl]-8-[1-[(4-fluorophenyl)methoxy]-1-methylethyl]-3,7-dihydro-(9Cl) Disclosed in WO00594449 on Oct. 12, 2000

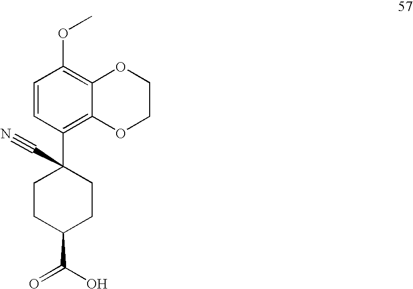

### 57 Clclohexanecarboxylic Acid, 4-cyano-4-(2,3-dihydro-8-methoxy-1,4-benzod ioxin-5-yl)-,cis(9Cl) Disclosed in WO 0014085 on Mar. 16, 2000.

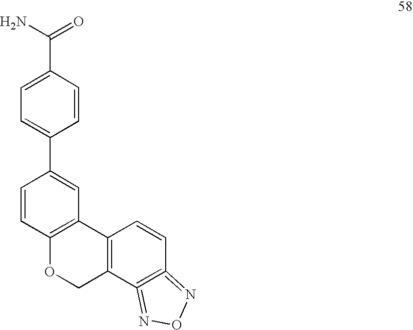

### 58 4-(7H-6,16-Dioxa-15,17-diaza-cyclopenta[a]phenanthren-2-yl)-benazamide

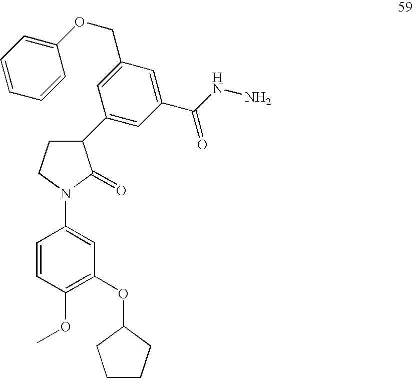

### 59 3-Benzyloxy-5-[1-(3-cyclopentyloxy-4-methoxy-phenyl)-2-oxo-pyrrolidin-3yl]-benzoic Acid Hydrazide

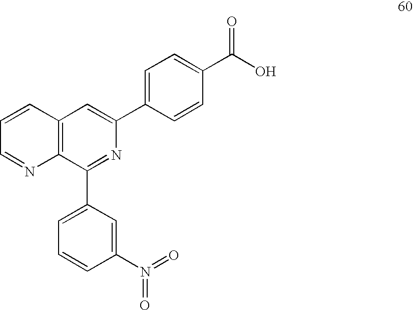

### 60 Benzoic Acid, 4-[8-(3-nitrophenyl)-1,7-naphthydrin-6-yl]-(9Cl) Disclosed in WO9818796 on May 7, 1998

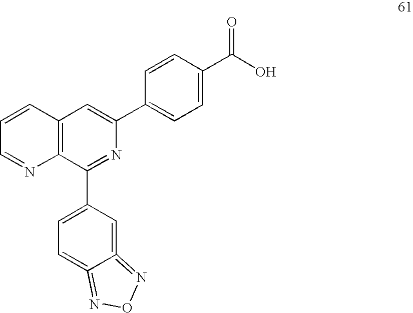

### 61 4-(8-Benzol[1,2,5] oxadiazol-5-yl-[1,7]napthyridin-6yl)-benzoic Acid

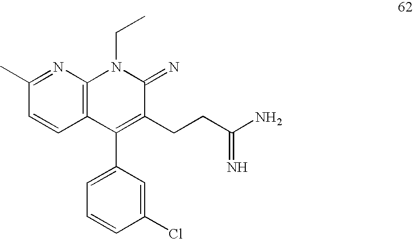

### 62 3-[4-(3-Chloro-phenyl)-1-ethyl-7-methyl-2-oxo-1,2,-dihdro-[1,8]naphthyridin-3-yl]-prpionamidine

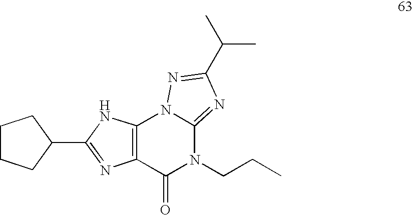

### 63 4H-[1,2,4]Triazole[5,1-b]purin-5(6H)-one, 7-cyclopentyl-2-(1methylethyl)-4-propyl (9Cl) Disclosed in WO 0035428 on Jun. 22, 2000

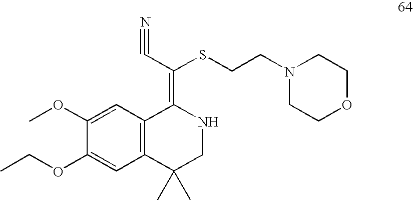

### 64 Acetronitrile, (6-ethoxy-3,4-dihydro-7-methoxy-4,4-dimethyl-1(2H)-isoquinolinylidene)[[2-(4-morpholinyl)ethyl[thio]-(9Cl) Disclosed in WO 0164647 on Sep. 7, 2001

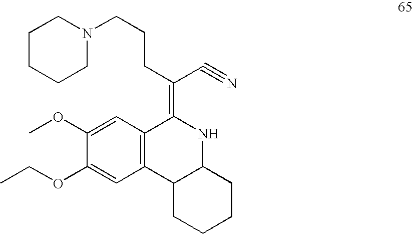

### 1-Piperidinepentanenitrile[(4aR, 1 ObR)-9-ethoxy-1,3,4,4a,5,10b-hexahydro-8-methoxy-6(2H)-phenanthridinylidene]-, rel-(9Cl) Disclosed in WO 164648 on Sep. 7, 2001

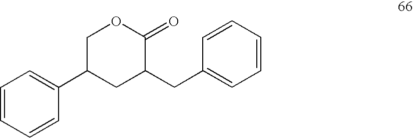

### 2H-Pyran-2-one,tetrahydro-5-phenyl-3-(phenylmethyl)-,trans-(9Cl) Disclosed in Chem, Pharm, Bull.(1992),40(9), 2525-30

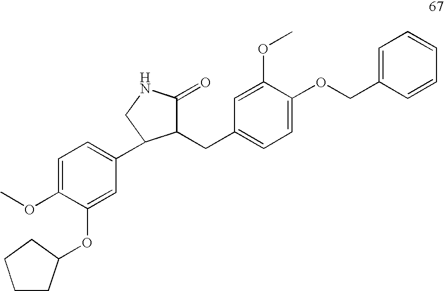

### 2-Pyrrolidinone, 4-[3-(cyclopentyloxy)-4-methoxyphenyl]-3-[[3-methoxy-4-(phenylmethoxy)phenyl]methyl]-(9Cl) Disclosed in WO 0168600 on Sep. 20, 2001.

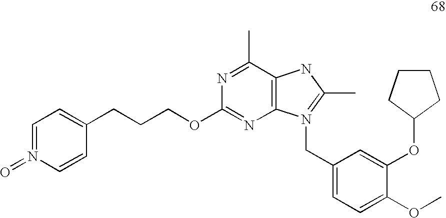

### 4-{3-[9-(3-Cyclopentyloxy-4-methoxy-benzyl)-6,8-dimethyl-9H-purin-2-yloxy]-propyl}-propyl}-pyridine 1-oxide

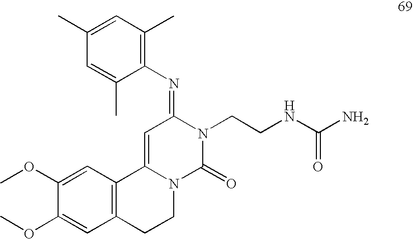

### Urea[2-[6,7-dihydro-9,10-dimethoxy-4-oxo-2-[(2,4,6-trimethylphenyl)imino]-2H-pyrimido[6,1-a]isoquinolin-3(4H)-yl]ethyl]-(9Cl) Disclosed in WO0058308 on Oct. 5, 2000

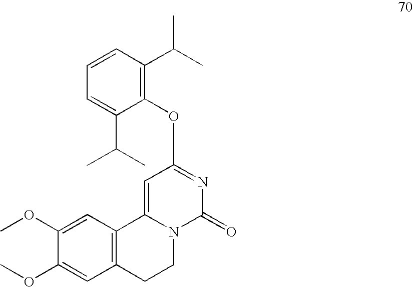

### 4H-Pyrimido[6,1-a]isoquinolin-4-one, 2-[2,6-bis(1-methylethyl)phenoxy]-6,6-dihydro-9,10-dimethoxy-(9Cl) Disclosed in WO 0058309 on Oct. 5, 2000

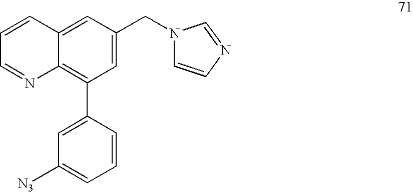

### 8-(3-Azido-phenyl)-6-imidazol-1-ylmeyhyl-quinoline

Another preferred PDEIV is a compound of the formula:

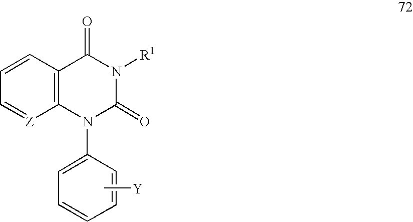

or a pharmaceutically-acceptable acid-addition salt thereof, wherein:

R₁ is hydrogen, alkyl of 1 to 3 carbon atoms, cyclopentylmethyl, cyclohexylmethyl, norbornylmethyl, [2.2.2]bicyclooctylmethyl or benzyl, the phenyl of the benzyl optionally being substituted by halogen; trifluoromethyl, nitor, carboxy or CO₂^{θ}M^{⊕} wherein M^{⊕} is a pharmaceutically aceptable cation;

Y is carboxy, carboalkoxy wherein the alkoxy has 1 to 6 carbon atoms, carbobenzyloxy, N-alkylcarboxamido wherein the alkyl has 1 to 6 carbon atoms, or CO₂^{θ}M^{⊕} wherein M^{⊕} is as defined above;

and Z is N or CH, provided that (i) when Z is CH, then R₁, is benzyl, Y is in the meta-position and Y may also be tetrazolyl optionally substituted by a group selected from alkyl of 1 to 3 carbon atoms and benzyl; (ii) when Z is N, Y is in the meta-or para-position of the 1-phenyl group and (iii) when R₁ is substituted benzyl, the substitution is at the meta-and/or para-positions and their pharmaceutically acceptable salts. Published in EP0260817 on May 15, 1991.

The terms “anxiolytic effective amount” and “antianxiety effective amount”, as used herein, refer to an amount that is effective in treating anxiety.

The term “antidepressant effective amount”, as used herein, refers to an amount that is effective in treating depression.

The term “treating” refers to, and includes, reversing, alleviating, inhibiting the progress of, or preventing a disease, disorder or condition, or one or more symptoms thereof; and “treatment” and “therapeutically” refer to the act of treating, as defined above.

The pharmaceutical compositions and methods of this invention comprise, or comprise administering PDE IV inhibitors of the formulas 1 through 72, which may have chiral centers and therefore exist in different enantiomeric forms. This invention includes methods and pharmaceutical compositions, as described above, wherein the PDE IV inhibitors that are employed are optical isomers, tautomers or stereoisomers of the compounds defined above, or mixtures thereof.

This present invention also relates to pharmaceutical compositions and methods comprising, or comprising administering, pharmaceutically acceptable acid addition salts of PDE IV inhibitors and of antidepressant and anxiolytic agents. The possible acids which are used to prepare the pharmaceutically acceptable acid addition salts of the basic active agents employed in the methods and pharmacuetical compositions of this invention are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3-naphthoate)]salts.

This invention also relates to pharmaceutical compositions and methods comprising, or comprising administering, pharmaceutically acceptable base addition salts of PDE IV inhibitors and of antidepressant and anxiolytic agents. The chemical bases that may be used as reagents to prepare pharmaceutically acceptable base salts of the acidic active agents that are employed in the methods of this invention are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine (meglumine), and the lower alkanolammonium and other base salts of pharmaceutically acceptable organic amines.

The subject invention also relates to pharmaceutical compositions and methods of treatment that employ isotopically-labeled compounds that are identical to those recited in PDE IV inhibitors, but for the fact that one or more atoms are replaced by an atom having an atomic mass or mass number different from the atomic mass or mass number usually found in nature. Examples of isotopes that can be incorporated into the PDE IV inhibitors that are employed in the pharmaceutical compositions and methods of the present invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹p, ³²P, ³⁵S, ¹⁸F, and ³⁶Cl, respectively. The PDE IV inhibitors employed in the pharmaceutical compositions and methods of the present invention, prodrugs thereof, and pharmaceutically acceptable salts of said compounds or of said prodrugs which contain the aforementioned isotopes and/or other isotopes are within the scope of this invention. Certain isotopically-labeled PDE IV inhibitors, for example, those into which radioactive isotopes such as ³H and ¹C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances.

### DETAILED DESCRIPTION OF THE INVENTION

The PDE IV inhibitors and antidepressants and anti-anxiety agents have been disclosed herein before in the application. The foregoing patents and patent applications are incorporated herein by reference in their entirety.

This invention relates both to methods of treating anxiety or depression in which the PDE IV inhibitors and the anxiolytic or antidepressant agent, or pharmaceutically acceptable salts of the same, are administered together, as part of the same pharmaceutical composition, as well as to methods in which these two active agents are administered separately as part of an appropriate dose regimen designed to obtain the benefits of the combination therapy. The appropriate dose regimen, the amount of each dose administered, and specific intervals between doses of each active agent will depend upon the subject being treated, the emetogen and the severity of the condition. Generally, in carrying out the methods of this invention, the PDE IV inhibitors will be administered to an adult human in an amount ranging from about 0.1 to about 30 mg/kg/day, in single or divided doses, preferably from about 0.5 to about 20 mg/kg/day. The compounds may be administered on a regimen of up to 6 times per day, preferably 1 to 4 times per day, especially 2 times per day and most especially once daily. A suitable dosage level for the antidepressant agent is about 0.5 to 1500 mg per day, preferably about 2.5 to 1000 mg per day, and especially about 2.5 to 500 mg per day. The compounds may be administered on a regimen of up to 6 time per day, preferably 1 to 4 times per day, especially 2 time per day and most especially once daily. A suitable dosage level for the anxiolytic agent is about 0.5 to 1500 mg per day, preferably about 2.5 to 1000 mg per day, and especially about 2.5 to 500 mg per day. The compounds may be administered on a regimen of up to 6 times per day, preferably 1 to 4 times per day, especially 2 times per day and most especially once daily. Variations may nevertheless occur depending upon the species of animal being treated and its individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effect, provided that such larger doses are first divided into several small doses for administration throughout the day.

The PDE IV inhibitors, their pharmaceutically acceptable salts, and the antidepressant and anxiolytic agents and their pharmaceutically acceptable salts that are employed in the pharmaceutical compositions and methods of this invention are hereinafter also referred to as “therapeutic agents”. The therapeutic agents can be administered via either the oral or parenteral route. Compositions containing both a PDE IV inhibitors and an anxiolytic or antidepressant agent, or pharmaceutically acceptable salts of one or both therapeutic agents, will generally be administered orally or parenterally daily, in single or divided doses, so that the total amount of each active agent administered falls within the above guidelines.

The therapeutic agents may be administered alone or in combination with pharmaceutically acceptable carriers or diluents by either of the routes previously indicated, and such administration may be carried out in single or multiple doses. More particularly, the therapeutic agents of this invention can be administered in a wide variety of different dosage forms, ie., they may be combined with various pharmaceutically acceptable inert carriers in the form of tablets, capsules, lozenges, troches, hard candies, suppositories, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the therapeutic agents of this invention, when administered separately (i.e., not in the same pharmaceutical composition) are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of a therapeutic agent in either sesame or peanut oil or in aqueous propylene glycol may be employed. The aqueous solutions should be suitably buffered if necessary and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art.

As stated above, the PDE IV inhibitors and the anxiolytic or antidepressant agent may be formulated in a single pharmaceutical composition or alternatively in individual pharmaceutical compositions for simultaneous, separate or sequential use in accordance with the present invention.

Preferably the compositions according to the present invention, which contain both a PDE IV inhibitors and an anxiolytic agent or an antidepressant, as well as the pharmaceutical compositions used to deliver only one of these active agents, are in unit dosage forms such as tablets, pills, capsules, powders, granules, solutions or suspensions, or suppositories, for oral, parenteral or rectal administration, by inhalation or insufflation or administration by transdermal patches or by buccal cavity absorption wafers.

For preparing solid compositions such as tablets, the principal active ingredient is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing, typically, from 0.05 to about 500 mg of each of the therapeutic agents contained in the composition. The tablets or pills of the composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac acetyl alcohol and cellulose acetate.

The liquid forms in which the novel compositions of the present invention may be incorporated for administration orally or by injection include aqueous solutions, suitably flavoured syrups, aqueous or oil suspensions, and flavored emulsions with edible oils such as cottonseed oil, sesame oil, coconut oil, peanut oil or soybean oil, as well as elixirs and similar pharmaceutical vehicles. Suitable dispersing or suspending agents for aqueous suspensions include synthetic and natural gums such as tragacanth, acacia, alginate, dextran, sodium carboxymethyl cellulose, methylcellulose, polyvinyl-pyrrolidone or gelatin.

Preferred compositions for administration of a PDE IV inhibitors or other therapeutic agent by injection include those comprising the therapeutic agent in association with a surface-active agent (or wetting agent or surfactant) or in the form of an emulsion (as a water-in-oil or oil-in-water emulsion).

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and preferably between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The therapeutic agent may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., eggs phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion will preferably comprise fat droplets between 0.1 and 1.0 μm, particularly 0.1 and 0.5 μm, and have a pH in the range of 5.5 to 8.0.

Compositions for inhalation or insufflation include solutions and suspensions in pharmaceutically acceptable, aqueous or organic solvents or mixtures thereof, and powders. The liquid or solid compositions may contain suitable pharmaceutically acceptable excipients as set out above. Preferably the compositions are administered by the oral or nasal respiratory route for local or systemic effect. Compositions in preferably sterile pharmaceutically acceptable solvents may be nebulised by use of inert gases. Nebulised solutions may be breathed directly from the nebulising device or the nebulising devise may be attached to a face mask, tent or intermittent positive pressure breathing machine. Solution, suspension, or powder compositions may be administered, preferably orally or nasally, from devices which deliver the formulation in an appropriate manner.

Compositions of the present invention may also be presented for administration in the form of transdermal patches using conventional technology. The compositions may also be administered via the buccal cavity using, for example, absorption wafers.

The present invention further provides a process for the preparation of a pharmaceutical composition comprising a PDE IV inhibitors and an antidepressant or anxiolytic agent, or pharmaceutically acceptable salts of the same, which process comprises bringing a PDE IV inhibitor and the antidepressant or anxiolytic agent (or the pharmaceutically acceptable salts of one or both of these therapeutic agents) into association with a pharmaceutically acceptable carrier or excipient.

It will be appreciated that the amount of the PDE IV inhibitors and the antidepressant or anxiolytic agent required for use in the treatment of depression or anxiety will vary not only with the particular compounds or compositions selected but also with the route of administration, the nature of the condition being treated, and the age and condition of the patient, and will ultimately be at the discretion of the patient's physician or pharmacist.

The antidepressant activity of the PDE IV compounds of the invention is determined by standard pharmacological tests including the behavioral despair paradigm described by R. D. Porsolt in Arch. Int. Pharmacodun. 227, 327(1997). The procedure comprises administering the compound to a mouse (Male CD (Charles River), weighing 20-25 g) which is then placed in a plexiglass cylinder (25 cm high and 10 cm in diameter) containing 6 cm water of 25° C. one hour after injection. The mouse is left in the cylinder for 6 minutes and after the first two minutes observed for duration of mobility.

The in vitro activity of the antidepressant or anxiolytic compounds used in this invention at the individual monoamine reuptake sites can be determined using rat synaptosomes or HEK-293 cells transfected with the human serotonin, dopamine or norepinephrine transporter, according to the following procedure adapted from those described by S. Snyder et al., (Molecular Pharmacology, 1971, 7, 66-80), D. T. Wong et al., (Biochemical Pharmacology, 1973, 22, 311-322), H. F. Bradford (Journal of Neurochemistry, 1969, 16, 675-684) and D. J. K. Balfour (European Journal of Pharmacology, 1973, 23, 19-26).

Synaptosomes: Male Sprague Dawley rats are decapitated and the brains rapidly removed. The cortex, hippocampi and corpus striata are dissected out and placed in ice cold sucrose buffer, 1 gram in 20 ml of buffer (the buffer is prepared using 320 mM sucrose containing 1 mg/ml glucose, 0.1 mM ethylenediamine tetraacetic acid (EDTA) adjusted to pH 7.4 with tris(hydroxymethyl)-aminomethane (TRIS) base). The tissues are homogenized in a glass homogenizing tube with a Teflon TM pestle at 350 rpm using a Potters homogenizer. The homogenate is centrifuged at 1000×g for 10 min. at 4° C. The resulting supernatant is recentrifuged at 17,000×g for 20 min. at 4° C. The final pellet is resuspended in an appropriate volume of sucrose buffer that yielded less than 10% uptake.

Cell Preparation: HEK-293 cells transfected with the human serotonin (5-HT), norepinephrine (NE) or dopamine (DA) transporter are grown in DMEM (Dulbecco's Modified Eagle Medium, Life Technologies Inc., 9800 Medical Center Dr., Gaithersburg, Md., catalog no. 11995-065)) supplemented with 10% dialyzed FBS (Fetal Bovine Serum, from Life Technologies, catalog no. 26300-053), 2 mM L-glutamine and 250 ug/ml G418 for the 5-HT and NE transporter or 2 ug/ml puromycin for the DA transporter, for selection pressure. The cells are grown in Gibco triple flasks, harvested with Phosphate Buffered Saline (Life Technologies, catalog no. 14190-136) and diluted to an appropriate amount to yield less than 10% uptake.

Neurotransmitter Uptake Assay: The uptake assays are conducted in glass tubes containing 50 uL of solvent, inhibitor or 10 uM sertraline, desipramine or nomifensine for the 5-HT, NE or DA assay nonspecific uptake, respectively. Each tube contains 400 uL of [3H]5-HT (5 nM final), [3H]NE (10 nM final) or [3H]DA (5 nM final) made up in modified Krebs solution containing 100 uM pargyline and glucose (1 mg/ml). The tubes are placed on ice and 50 uL of synaptosomes or cells is added to each tube. The tubes are then incubated at 370 C for 7 min. (5-HT, DA) or 10 min. (NE). The incubation is terminated by filtration (GF/B filters), using a 96-well Brandel Cell Harvester, the filters are washed with modified Krebs buffer and counted using either a Wallac Model 1214 or Wallac Beta Plate Model 1205 scintillation counter.

Determination of the in vivo serotonin reuptake inhibition activity and potency of action for the compounds of the present invention can be made by measuring the ability of the compound to block the depletion of serotonin in the anterior cortex induced by (+/−)-para-chloroamphetamine (PCA) in the rat, according to a procedure adapted from R. W. Fuller, H. D. Snoddy and M. L. Cohen in Neuropharmacology 23: 539-544 (1984).

Generally, male, white Sprague-Dawley rats weighing 160-230 9 each are assigned to either the control (vehicle) or test groups. When the test compound is administered subcutaneously (sc) at a given dose, it is co-administered with 5 mg/kg of para-chloroamphetamine (PCA). Three hours post-dose, the animals are sacrificed by decapitation and the anterior cortices are removed, wrapped in parafilm and frozen in dry ice (−78 C). When dosed orally (po), the rats are fasted the night before the experiment and then treated with the test compound at a given dose 30 minutes prior to the administration of the PCA (5 mg/kg, sc). After three hours, the animals are sacrificed and the tissues removed as above.

To determine the serotonin (5-HT) levels, the frozen tissues are homogenized with Branson sonifier in 0.5 mL of mobile phase in Eppendorf centrifuge tubes. Samples are then spun down at 11000 rpm for twenty minutes in a Sorval SH-MT rotor in a Sorval RC5C centrifuge. The supernatant thus obtained is pipetted into HPLC vials and the 5-HT levels are measured on HPLC-EC.

Interpretation of the results is as follows: Each experiment has a set of vehicle treated animals and a set of PCA-only animals. The mean 5-HT value of the PCA animals is subtracted from the mean 5-HT value of the vehicle animals. This is the signal or window of the response. The mean 5-HT value of each test group is determined, the mean of the PCA group subtracted from that, and that amount divided by the window is the percent protection from the PCA effect for that dose. To report an ID₅₀, a line is drawn mathematically through the percent protection values and the 50 percent level calculated.

All of the antidepressants or anxiolytic compounds were assayed in vitro for serotonin, dopamine, and norepinephrine reuptake inhibition, and all had IC₅₀ values of about less than or equal to 250 nM for serotonin reuptake inhibition, about less than or equal to 1000 nM for dopamine reuptake inhibition, and about less than or equal to 1000 nM for norepinephrine reuptake inhibition.

When administered in combination, either as a single or as separate pharmaceutical composition(s), a PDE IV inhibitor and an antidepressant or anxiolytic agent, are presented in a ratio which is consistent with the manifestation of the desired effect. In particular, the ratio by weight of the PDE IV inhibitor and the anxiolytic or antidepressant agent will suitably be between 0.001 to 1 and 1000 to 1, and especially between 0.01 to 1 and 100 to 1.

As used herein the term “mammal” includes animals of economic importance such as bovine, ovine, and porcine animals, especially those that produce meat, as well as domestic animals (e.g. cats and dogs), sports animals (e.g. horses), zoo animals, and humans, the latter being preferred.

1. A pharmaceutical composition for the treatment of anxiety or depression in a mammal, comprising: (a) a compound that exhibits activity, respectively, as an antidepressant or anxiolytic agent, or a pharmaceutically acceptable salt thereof; (b) a PDE IV inhibitor or pharmaceutically acceptable salt thereof; and (c) a pharmaceutically acceptable carrier; wherein the active agents “a” and “b” above are present in amounts that render the composition effective in treating, respectively, anxiety or depression with increased efficacy.

2. A pharmaceutical composition according to claim 1, wherein the antidepressant agent or anxiolytic agent or pharmaceutically acceptable salt thereof is selected from the group consisting of the following agents and their pharmaceutically acceptable salts:
citalopram;
fluoxetine;
sertraline;
paroxetine;
nefazadone;
bupropion;
escitalopram;
zimelidine;
fluvoxamine;
duloxetine;
milnacipran;
venlafaxine;
trazodone;
mirtazapine;
amitriptyline;
imipramine;
lubazodone hydrochloride;
[Morpholine, 2-[[(7-fluoro-2,3-dihydro-1H-inden-4yl)oxy]methyl]-, hydrochloride, (2S)-(9CL];
2-benzofuran-carboxamide, 5-[4-[4-(5-cyano-1 h-indol-3-yl)butyl]-1-piperazinyl]-(9Cl);
mianserin;
tianeptine;
minaprine;
phenelzine (MAO-I);
isocarboxazid (MAO-I);
tranylcypromine (MAO-I) and
St John's Wort:

3. A pharmaceutical composition according to claim 1 wherein a PDE IV inhibitor or a pharmaceutically acceptable salt thereof is selected from the group consisting of:
a. cilomilast;
b. roflumilast;
c. BAY 19-8004 [2-(2,4-Dichloro-benzoyl-6-methanesulfonyl-benzofuran-3-yl]-urea;
d. pumafentrine;
e. V-11294A 3H-Purin-6-amine 3-[(3-cyclopentyloxy-)-4-methoxy-phenyl]methyl]-N-ethyl-8-(1-methylethyl-, monohydrochloride;
f. CDC-801 2H-Isoindole-2-propan-amide B-[3-cyclopentoxy)-4 methoxyphenyl]-1,3-dihydro-1,3-cloxo-(9Cl);
g. cipamfylline;
h. mesopram;
i. SCH-351591-5-Quinolinecarboxamide, N-(3,5-dichloro-1-oxido-4-pyridinyl)-8-methoxy-2-(trifluorometomethyl)-(9Cl);
j. YM-976 Pyrido[2,3-d]pyrimidin-2(1H)-one, 4-(3-chloro-phenyl)-1,7-diefyl-(9Cl);
k. Cl-1044 3-pyridine carboxamide, N-(9-amino-3,4,6,7-tetrahydro-4-oxo-1-phenyl pyrrolo[3,2,1 jk] [1,4]benzodiazepin-3-yl)(R)-(9Cl);
l. Cyclohexanol 4-[4-2-amino-5-pyrimidinyl)phenyl]-4-3-(cyclo-pentyloxy)-4-methoxylphenyl]-trans-(9Cl);
m. Cyclohexanol, 4-[(2-amino-5-pyrinidinyl, ethynly]-4-[3-(cyclopentoxy)-4-methoxypheny]-cis-(9Cl);
n. 4-(3-sec-Butoxy-4-methoxy-phenyl-4-(3-[1,2,4]oxadiazol-5-yl-phenylethynyl)-cyclohexanol;
o. 6-(3-Cyclopropylmethoxy-4-methoxymethyl-phenyl-8-methoxy-9-methoxy-methyl-1,2,3,4,4a, 10b-hexahydro-phenanthridine;
p. 4-(7-Methoxy-2,2-dimethyl-2-3-dihydro-benzofuran-4-yl)-2-[4-(4-methyl-6-oxo-1,4,5,6-tetrahydro-pyridazin-3-yl)-phenyl]-4a,5,8,8a-tetrahydro-2H-phthalazin-1-one;
q. Morpholine, 4-[[4-[(4aR,8aS)-4-(2,3-dihydro-7-methoxy-2,2-dimethyl-4-benzofuranyl)-4a,5,8,8a-tetrahydro-1-oxo-2(1H)-phthalazinyl]phenyl]sulfonyl]-,rel-(9Cl);
r. 1(2H)-Phthalazinone, 4-(2,3-dihydro-7-methoxy-2,2-dimethyl-4-benzofuranyl)-4a,5,8,8a-tetrahydro-2-(tetrahydro-2H-thiopyran-4-yl)-, (4aR,8aS)-rel-(9Cl); 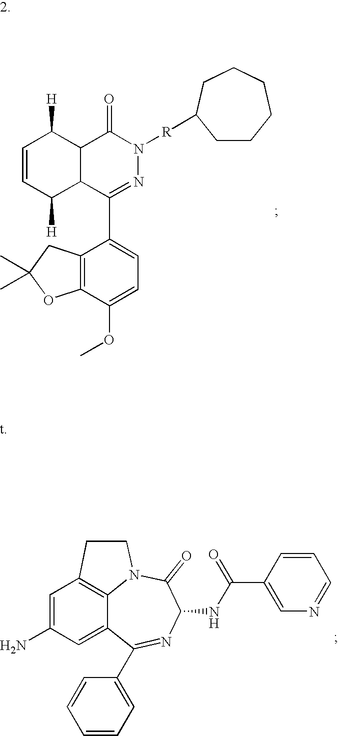
u. Tofimilast 5H-Pyrazolo[3,4-c]-1,2,4-triazolo[4,3-a]pyridine, 9-cyclopentyl-7-ethyl-6,9-dihydro-3-(2-thienyl)-(9Cl);
v. 5-Pyrimidinecarboxamide, 4-(1,3-benzodioxol-5-yloxy)-N-[[2-fluoro-4(1-hydroxyl-methyl ethyl)phenyl]methyl]-(9Cl);
w. 2-(Benzo[1,2,5]oxadiazol-5-yloxy)-N-[4-(1-hydroxy-1-methyl-ethyl)-benzyl]-nicotinamide;
x. [1,2,4]Triazolo[4,3-a]quinazolin-5(4H)-one,7-bromo-1-(dimethylamino)-4-[3-(3-pyridinyl)-2-propenyl]-(9Cl);
y. Cyanamide, [1-ethyl-1,6-dihydro-3-(1-methylethyl)-5-phenylpyrazolo[4,3-e][1,4]diazepin-8-yl-(9Cl);
z. 2-pyrrolidinone, 4-[3-cyclopentyloxy)-4-methoxyphenyl]-(9Cl);
i. 1-Pyrrolidinecarboxylic acid, 4-[3-(cyclopentyloxy)-4-methoxyphenyl]3-formyl-3-methyl-,methyl ester, (3S,4S)-(9Cl);
ii. 3-Pyrrolidinemethanamine, 4-[[3-(cyclopentyloxy)-4-methoxyphenyl]-N,3-dimethyl-1-(phenylmethyl)-, (3R,4S)-(9Cl);
aa. [4-(1-Cyclopentyl-3-ethyl-1H-indazol-6-yl)-3-methyl-1-(1-phenyl-ethyl)-pyrrolidin-3-yl]-methanol;
bb. 1-Pyrrolidinecarboxylic acid, 4-[3-(cyclopentyloxy)-4-methoxyphenyl]-3-methyl-3-[1-(methylhydrazono)ethyl]-,methyl ester (9Cl);
cc. 1H-Pyrazole-4-carboxylic acid, 1-cyclohexyl-3,5-dimethyl-,ethyl ester (9Cl);
dd. 1H-Pyrrole-3-carboxylic acid, 2-methyl-1-(3-nitrophenyl)-5-phenyl-, ethyl ester (9Cl);
ee. Pyridine, 4-[2-[3-(cyclopentyloxy)-4-methoxyphenyl]-2-phenylethyl]-(9Cl);
ff. Benzenemethanol, 4-[1-[3,4-bis(difluoromethoxy)phenyl]-2-(1-oxido-4-pyridinyl)ethyl]-α,α-bis(trifluoromethyl)-(9Cl);
gg. 2-{4-[1-(3,4-Bis-difluoromethoxy-phenyl)-2-(3-methyl-1-Oxy-pyridin-4-yl)-ethyl]-phenyl}-1,1,1,3,3,3-hexafluoro-proppane;
i. -2-{4-[1-(3-Cyclobutyloxy-4-difluoromethoxy-phenyl)-2(3-methyl-1-oxy-pyridin-4-yl)-ethyl]-phenyl}-1,1,1,3,3,3-hexfluoro-propan-2-ol;
ii. 2-{4-[1-(3-Cyclobutyloxy-4-difluoromethoxy-phenyl)-2-(1-oxy-pyridin-4-yl-ethyl]-phenyl}-1,1,1,3,3,3-hexafluoro-propan-2-ol;
hh. 2-Pyridinamine,5-[1-[3,4-bis(difluormethoxy)phenyl]-2(4-pyridinyl)ethyl]-N-(phenylmethyl)-(9Cl);
ii. 2-(5-[1-(3,4-Bis-difluoromethoxy-phenyl)-2-(1-oxy-pyridin-4-yl)-ethyl]-thiazol-2-yl}-propan-2-ol;
jj. 6-isopropyl-8-{3-[2-(4-methanesulfonyl-phenyl)-2-phenyl-ethyl-phenyl}quinoline;
kk. 1H-Indole-2-carboxamide, 1-[(4-flurophenyl)methyl]-3-(phenylmethoxy)-N-3-pyridinyl-(9Cl);
ll. 4-Difluoromethoxy-2-methyl-2,3-dihydro-benzooxazole-7-carboxylic acid (3,5-dimethyl-isoxazol-4-yl)-amide;
mm. 2-Acetyl-4-difluoromethoxy-benezooxazole-7-carboxylic acid(3,5-dichloro-pyridin-4-yl-amide;
nn. 1H-Isoindole-1,3)2H)-dione,2-[1-[3-(cyclopentyloxy)-4-methoxyphenyl]-2-(1,3,4-oxadiazol-2-yl)ethyl]-5-methyl-(9Cl);
oo. Benezenemethanamine,N-[3-[1-[(3,5-dichloro-4-pyridinyl)methyl]-6-methoxy-5-phthalazinyl]-2-propynyl]-N-methyl-(9Cl);
pp. 8-Cyclopentyloxy-4-(3,5-dichloro-pyridin-4-ylmethyl)-2-methanesulfonyl-7-methoxy-1,2-dihydro-phthalazine;
qq. 1,2,4-Triazole[3,4-alphthalazine,6-[3,5-dichloro-4-pyridinyl)methyl]-9-methoxy-3-methyl-(9Cl);
rr. Isoquinoline,5-(cyclopentylmethyl)-1-[(3,5-dicloro-4-pyridinyl)methyl]-3-4-dihydro-6-methoxy-(9Cl);
i. 1-(3,5-Dichloro-pyridin-4-ylmethyl)-6-methoxy-5-thiazol-2-ymethyl-phthalazine;
ii. 1-(3,5-Dichloro-pyridin-4-ylmethyl)-6-methoxy-5-(5H-[1,2,4]triazol-1-ylmethyl)-phthalazine; 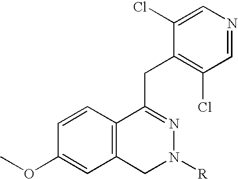
48 R=SO₂CH₃
49 R=COCH₂Ph Phthalazine, 4-[(3,5-dichloro-4-pyridinyl)methyl]-1,2-dihydro-7-methoxy-2-(phenlacetyl)-(9Cl)
ss. {4-[3-(3-Ethoxy-4-methoxy-phenyl)-5,6-dihydro-4H-pyridazine-1-carbonyl}-carbamic acid methyl ester;
tt. 4-Pyridinecarboxamide, N-[4-[[3-(3-ethoxy-4-methoxyphenyl)-5,6-dihydro-1(4H)-Pyridazinyl]carbonyl]phenyl-(9Cl);
uu. 1-{4-[3-(3-Ethoxy-4-methoxy-phenyl)-5.6-dihydro-4H-pyridazine-1-carbonyl]-phenyl}-3-methyl-urea;
vv. Urea, [2-(2,4-d ichlorobenzoyl)-6-[(3E)-3-pentenyloxy]-3-benzofuranyl]-(9Cl);
ww. Benzene sulfonic acid, 4-[(dimethylamino)sulfonyl]amino],-3-[(aminocarbonyl)amino]-2-(2,4,-dichlorobenzoyl)-6-benzofuranyl ester (9Cl);
xx. Urea,[2-(cyclohexylcarbonyl)-6-methoxy-3-benzofuranyl]-(9Cl);
yy. 6H-Purin-6-one,3-[[3-(cyclopentyloxy)-4methoxyphenyl]methyl]-8-[1-[(4-fluorophenyl)methoxy]-1-methylethyl]-3,7-dihydro-(9Cl);
zz. Clclohexanecarboxylic acid,4-cyano-4-(2,3-dihydro-8-methoxy-1,4-benzodioxin-5-yl)-,cis(9Cl);
aaa. 4-(7H-6,16-Dioxa-15,17-diaza-cyclopenta[a]phenanthren-2-yl)-benazamide;
bbb. 3-Benzyloxy-5-[1-(3-cyclopentyloxy-4-methoxy-phenyl)-2-oxo-pyrrolidin-3yl]-benzoic acid hydrazide;
ccc. Benzoic acid, 4-[8-(3-nitrophenyl)-1,7-naphthydrin-6-yl]-(9Cl);
ddd. 4-(8-Benzol[1,2,5] oxadiazol-5-yl-[1,7]napthyridin-6yl)-benzoic acid;
eee. 3-[4-(3-Chloro-phenyl)-1-ethyl-7-methyl-2-oxo-1,2,-dihdro-[1,8]naphthyridin-3-yl]-prpionamidine;
fff. 4H-[1,2,4]Triazole[5, 1-b]purin-5(6H)-one, 7-cyclopentyl-2-(1methylethyl)-4-propyl (9Cl);
ggg. Acetronitrile, (6-ethoxy-3,4-dihydro-7-methoxy-4,4-dimethyl-1(2H)-isoquinolinylidene)[[2-(4-morpholinyl)ethyl[thio]-(9Cl);
hhh. 1-Piperidinepentanenitrile[(4aR, 10bR)-9-ethoxy-1,3,4,4a,5,10b-hexahydro-8-methoxy-6(2H)-phenanthridinylidene]-, rel-(9Cl);
iii. 2H-Pyran-2-one,tetrahydro-5-phenyl-3-(phenylmethyl)-,trans-(9Cl);
jjj. 2-Pyrrolidinone, 4-[3-(cyclopentyloxy)-4-methoxyphenyl]-3-[[3-methoxy-4-(phenylmethoxy)phenyl]methyl]-(9Cl);
kkk. 4-{3-[9-(3-Cyclopentyloxy-4-methoxy-benzyl)-6,8-dimethyl-9H-purin-2-yloxy]-propyl}-propyl}-pyridine 1-oxide;
lll. Urea[2-[6,7-dihydro-9,10-dimethoxy-4-oxo-2-[(2,4,6-trimethylphenyl)imino]-2H-pyrimido[6, 1-a]isoquinolin-3(4H)-yl]ethyl]-(9Cl);
mmm. 4H-Pyrimido[6,1-a]isoquinolin-4-one,2-[2,6-bis(1-methylethyl)phenoxy]-6,6-dihydro-9,10-dimethoxy-(9Cl);
nnn. 8-(3-Azido-phenyl)-6-imidazol-1-ylmeyhyl-quinoline; and
ooo. 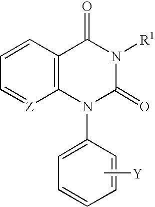
wherein:
R₁ is hydrogen, alkyl of 1 to 3 carbon atoms, cyclopentylmethyl, cyclohexylmethyl, norbornylmethyl, [2.2.2]bicyclooctylmethyl or benzyl, the phenyl of the benzyl optionally being substituted by halogen; trifluoromethyl, nitor, carboxy or CO₂^{θ}M^{⊕} wherein M^{⊕} is a pharmaceutically aceptable cation;
Y is carboxy, carboalkoxy wherein the alkoxy has 1 to 6 carbon atoms, carbobenzyloxy, N-alkylcarboxamido wherein the alkyl has 1 to 6 carbon atoms, or CO₂^{θ}M^{⊕} wherein M^{⊕} is as defined above;
and Z is N or CH, provided that (i) when Z is CH, then R₁, is benzyl, Y is in the meta-position and Y may also be tetrazolyl optionally substituted by a group selected from alkyl of 1 25 to 3 carbon atoms and benzyl; (ii) when Z is N, Y is in the meta-or para-position of-the 1-phenyl group and (iii) when R₁ is substituted benzyl, the substitution is at the meta-and/or para-positions.

4. A pharmaceutical composition according to claim 1 wherein the amount of the antidepressant or anxiolytic agent, or pharmaceutically acceptable salt thereof, in said composition is from about 0.05 mg to about 1500 mg and the amount of the PDE IV inhibitor or pharmaceutically acceptable salt thereof is from about 0.1 mg/kg/day to about 30 mg/kg/day.

5. A pharmaceutical composition according to claim 3 wherein the amount of the anxiolytic agent or antidepressant, or pharmaceutically acceptable salt thereof, in said composition is from about 2.5 mg to about 500 mg and the amount of the PDE IV inhibitor or pharmaceutically acceptable salt thereof is from about 0.5 mg/kg/day to about 20 mg/kg/day.

6. A method of treating anxiety or depression in a mammal, comprising administering to said mammal: (a) a compound that exhibits activity as an antidepressant or anxiolytic agent, or a pharmaceutically acceptable salt thereof; and (b) a PDE IV inhibitor or pharmaceutically acceptable salt thereof; wherein the active agents “a” and “b” above are present in amounts that render the combination of the two agents effective in treating, respectively, anxiety or depression with increased efficacy.

7. The method according to claim 6, wherein the anxiolytic or antidepressant or pharmaceutically acceptable salt thereof is selected from the group consisting of:
citalopram
fluoxetine
sertraline
paroxetine
nefazadone
bupropion
escitalopram
zimelidine
fluvoxamine
duloxetine
milnacipran
venlafaxine
trazodone
mirtazapine
amitriptyline
imipramine
lubazodone hydrochloride;
[Morpholine, 2-[[(7-fluoro-2,3-dihydro-1H-inden-4yl)oxy]methyl]-, hydrochloride, (2S)-(9Cl]
2-Benzofuran-carboxamide, 5-[4-[4-(5-cyano-1H-indol-3-yl)butyl]-1-piperazinyl]-(9Cl) mianserin
tianeptine
minaprine
pheneizine (MAO-I)
isocarboxazid (MAO-I)
tranylcypromine (MAO-I)
St John's Wort

8. The method according to claim 6, wherein the anxiolytic or antidepressant, or pharmaceutically acceptable salt thereof, and the PDE IV inhibitor or pharmaceutically acceptable salt thereof, are administered as part of the same dosage form.

9. The method according to claim 6, wherein the PDE IV inhibitor, or pharmaceutically acceptable salt thereof, is administered in an amount from about 0.1 mg/kg/day to about 30 mg/kg/day per day, and the anxiolytic agent or antidepressant, or pharmaceutically acceptable salt thereof, is administered in an amount from about 0.05 mg day to about 1500 mg per day.

10. The method according to claim 6, wherein the PDE IV inhibitor is administered in an amount ranging from about 0.5 mg/kg/day to about 20 mg/kg/day and the SR1 is administered in an amount ranging from about 2.5 mg per day to 500 mg per day.

11. The method according to claim 6, wherein the PDE IV inhibitor or a pharmaceutically acceptable salt thereof is selected from:
a. cilomilast;
b. roflumilast;
c. BAY 19-8004 [2-(2,4-Dichloro-benzoyl-6-methanesulfonyl-benzofuran-3-yl]-urea;
d. pumafentrine;
e. V-11294A 3H-Purin-6-amine 3-[(3-cyclopentyloxy-)-4-methoxy-phenyl]methyl]-N-ethyl-8-(1-methylethyl-, monohydrochloride;
f. CDC-801 2H-Isoindole-2-propan-amide B-[3-cyclopentoxy)-4 methoxyphenyl]-1,3-dihydro-1,3-cloxo-(9Cl);
g. cipamfylline;
h. mesopram;
i. SCH-351591-5-Quinolinecarboxamide, N-(3,5-dichloro-1-oxido-4-pyridinyl)-8-methoxy-2-(trifluorometomethyl)-(9Cl);
j. YM-976 Pyrido[2,3-d]pyrimidin-2(1H)-one, 4-(3-chloro-phenyl)-1,7-diefyl-(9Cl);
k. Cl-1044 3-pyridine carboxamide, N-(9-amino-3,4,6,7-tetrahydro-4-oxo-1-phenyl pyrrolo[3,2,1-jk] [1,4]benzodiazepin-3-yl)(R)-(9Cl);
l. Cyclohexanol 4-[4-2-amino-5-pyrimidinyl)phenyl]-4-3-(cyclo-pentyloxy)-4-methoxy]phenyl]-trans-(9Cl);
m. Cyclohexanol, 4-[(2-amino-5-pyrinidinyl, ethynly]-4-[3-(cyclopentoxy)-4-methoxypheny]-cis-(9Cl);
n. 4-(3-sec-Butoxy-4-methoxy-phenyl-4-(3-[1,2,4]oxadiazol-5-yl-phenylethynyl)-cyclohexanol;
o. 6-(3-Cyclopropylmethoxy-4-methoxymethyl-phenyl-8-methoxy-9-methoxy-methyl-1,2,3,4,4a,10b-hexahydro-phenanthridine;
p. 4-(7-Methoxy-2,2-dimethyl-2-3-dihydro-benzofuran-4-yl)-2-[4-(4-methyl-6-oxo-1,4,5,6-tetrahydro-pyridazin-3-yl)-phenyl]-4a,5,8,8a-tetrahydro-2H-phthalazin-1-one;
q. Morpholine, 4-[[4-[(4aR,8aS)-4-(2,3-dihydro-7-methoxy-2,2-dimethyl-4-benzofuranyl)-4a,5,8,8a-tetrahydro-1-oxo-2(1H)-phthalazinyl]phenyl]sulfonyl]-,rel-(9Cl);
r. 1 (2H)-Phthalazinone, 4-(2,3-dihydro-7-methoxy-2,2-dimethyl-4-benzofuranyl)-4a,5,8,8a-tetrahydro-2-(tetrahydro-2H-thiopyran-4-yl)-, (4aR,8aS)-rel-(9Cl);
s. 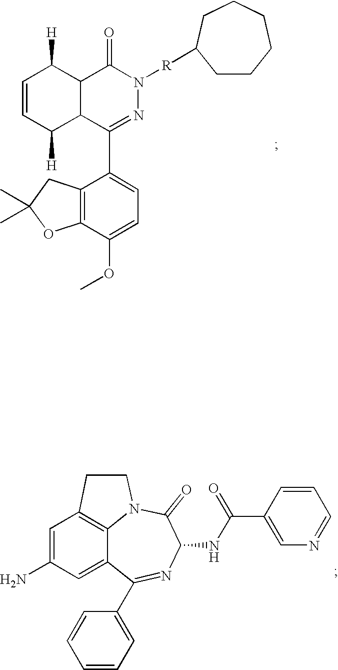
t.
u. Tofimilast 5H-Pyrazolo[3,4-c]-1,2,4-triazolo[4,3-a]pyridine, 9-cyclopentyl-7-ethyl-6,9-dihydro-3-(2-thienyl)-(9Cl);
v. 5-Pyrimidinecarboxamide, 4-(1,3-benzodioxol-5-yloxy)-N-[[2-fluoro-4(1-hydroxy-1-methylethyl)phenyl]methyl]-(9Cl);
w. 2-(Benzo[1,2,5]oxadiazol-5-yloxy)-N-[4-(1-hydroxy-1-methyl-ethyl)-benzyl]-nicotinamide;
x. [1,2,4]Triazolo[4,3-a]quinazolin-5(4H)-one,7-bromo-1-(dimethylamino)-4-[3-(3-pyridinyl)-2-propenyl]-(9Cl);
y. Cyanamide, [1-ethyl-1,6-dihydro-3-(1-methylethyl)-5-phenylpyrazolo[4,3-e][1,4]diazepin-8-yl]-(9Cl);
z. 2-pyrrolidinone, 4-[3-cyclopentyloxy)-4-methoxyphenyl]-(9Cl);
i. 1-Pyrrolidinecarboxylic acid, 4-[3-(cyclopentyloxy)-4-methoxyphenyl]3-formyl-3-methyl-,methyl ester, (3S,4S)-(9Cl);
ii. 3-Pyrrolidinemethanamine, 4-[[3-(cyclopentyloxy)-4-methoxyphenyl]-N,3-dimethyl-1-(phenylmethyl)-, (3R,4S)-(9Cl);
aa. [4-(1-Cyclopentyl-3-ethyl-1H-indazol-6-yl)-3-methyl-1-(1-phenyl-ethyl)-pyrrolidin-3-yl]-methanol;
bb. 1-Pyrrolidinecarboxylic acid, 4-[3-(cyclopentyloxy)-4-methoxyphenyl]-3-methyl-3-[1-(methylhydrazono)ethyl]-,methyl ester (9Cl);
cc. 1H-Pyrazole-4-carboxylic acid, 1-cyclohexyl-3,5-dimethyl-,ethyl ester (9Cl);
dd. 1H-Pyrrole-3-carboxylic acid, 2-methyl-1-(3-nitrophenyl)-5-phenyl-, ethyl ester (9Cl);
ee. Pyridine, 4-[2-[3-(cyclopentyloxy)-4-methoxyphenyl]-2-phenylethyl]-(9Cl);
ff. Benzenemethanol, 4-[1-[3,4-bis(difluoromethoxy)phenyl]-2-(1-oxido-4-pyridinyl)ethyl]-α,α-bis(trifluoromethyl)-(9Cl);
gg. 2-{4-[1-(3,4-Bis-difluoromethoxy-phenyl)-2-(3-methyl-1-Oxy-pyridin-4-yl)-ethyl]-phenyl}-1,1,1,3,3,3-hexafluoro-proppane;
i. -2-{4-[1-(3-Cyclobutyloxy-4-difluoromethoxy-phenyl)-2(3-methyl-1-oxy-pyridin-4-yl)-ethyl]-phenyl}-1,1,1, 3,3,3-hexfluoro-propan-2-ol;
ii. 2-{4-[1-(3-Cyclobutyloxy-4-difluoromethoxy-phenyl)-2-(1-oxy-pyridin-4-yl-ethyl]-phenyl}-1,1,3,3,3-hexafluoro-propan-2-ol;
hh. 2-Pyridinamine,5-[1-[3,4-bis(difluormethoxy)phenyl]-2(4-pyridinyl)ethyl]-N-(phenylmethyl)-(9Cl);
ii. 2-{5-[1-(3,4-Bis-difluoromethoxy-phenyl)-2-(1-oxy-pyridin-4-yl)-ethyl]-thiazol-2-yl}-propan-2-ol;
jj. 6-Isopropyl-8-{3-[2-(4-methanesulfonyl-phenyl)-2-phenyl-ethyl-phenyl}quinoline;
kk. 1H-Indole-2-carboxamide, 1-[(4-flurophenyl)methyl]-3-(phenylmethoxy)-N-3-pyridinyl-(9Cl);
ll. 4-Difluoromethoxy-2-methyl-2,3-dihydro-benzooxazole-7-carboxylic acid (3,5-dimethyl-isoxazol-4-yl)-amide;
mm. 2-Acetyl-4-difluoromethoxy-benezooxazole-7-carboxylic acid(3,5-dichloro-pyridin-4-yl-amide;
nn. 1H-Isoindole-1,3)2H)-dione,2-[1-[3-(cyclopentyloxy)-4-methoxyphenyl]-2-(1,3,4-oxadiazol-2-yl)ethyl]-5-methyl-(9Cl);
oo. Benezenemethanamine, N-[3-[1-[(3,5-dichloro-4-pyridinyl)methyl]-6-methoxy-5-phthalazinyl]-2-propynyl]-N-methyl-(9Cl);
pp. 8-Cyclopentyloxy-4-(3,5-dichloro-pyridin-4-ylmethyl)-2-methanesulfonyl-7-methoxy-1,2-dihydro-phthalazine;
qq. 1,2,4-Triazole[3,4-a]phthalazine,6-[3,5-dichloro-4-pyridinyl)methyl]-9-methoxy-3-methyl-(9Cl);
rr. Isoquinoline,5-(cyclopentylmethyl)-1-[(3,5-dicloro-4-pyridinyl)methyl]-3-4-dihydro-6-methoxy-(9Cl);
i. 1-(3,5-Dichloro-pyridin-4-ylmethyl)-6-methoxy-5-thiazol-2-ylmethyl-phthalazine;
ii. 1-(3,5-Dichloro-pyridin-4-ylmethyl)-6-methoxy-5-(5H-[1,2,4]triazol-1-ylmethyl)-phthalazine; 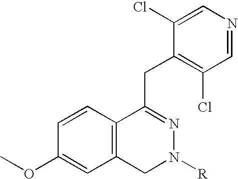
48 R=SO₂CH₃
49 R=COCH₂Ph Phthalazine, 4-[(3,5-dichloro-4-pyridinyl)methyl]-1,2-dihydro-7-methoxy-2-(phenlacetyl)-(9Cl)
ss. {4-[3-(3-Ethoxy-4-methoxy-phenyl)-5,6-dihydro-4H-pyridazine-1-carbonyl}-carbamic acid methyl ester;
tt. 4-Pyridinecarboxamide, N-[4-[[3-(3-ethoxy-4-methoxyphenyl)-5,6-dihydro-1 (4H)-Pyridazinyl]carbonyl]phenyl-(9Cl);
uu. 1-{4-[3-(3-Ethoxy-4-methoxy-phenyl)-5.6-dihydro-4H-pyridazine-1-carbonyl]-phenyl}-3-methyl-urea;
vv. Urea, [2-(2,4-dichlorobenzoyl)-6-[(3E)-3-pentenyloxy]-3-benzofuranyl]-(9Cl);
ww. Benzene sulfonic acid, 4-[(dimethylamino)sulfonyl]amino],-3-[(aminocarbonyl)amino]-2-(2,4,-dichlorobenzoyl)-6-benzofuranyl ester (9Cl);
xx. Urea,[2-(cyclohexylcarbonyl)-6-methoxy-3-benzofuranyl]-(9Cl);
yy. 6H-Purin-6-one,3-[[3-(cyclopentyloxy)-4methoxyphenyl]methyl]-8-[1-[(4-fluorophenyl)methoxy]-1-methylethyl]-3,7-dihydro-(9Cl);
zz. Clclohexanecarboxylic acid,4-cyano-4-(2,3-dihydro-8-methoxy-1,4-benzodioxin-5-yl)-,cis(9Cl);
aaa. 4-(7H-6,16-Dioxa-15,17-diaza-cyclopenta[a]phenanthren-2-yl)-benazamide;
bbb. 3-Benzyloxy-5-[1-(3-cyclopentyloxy-4-methoxy-phenyl)-2-oxo-pyrrolidin-3yl]-benzoic acid hydrazide;
ccc. Benzoic acid, 4-[8-(3-nitrophenyl)-1,7-naphthydrin-6-yl]-(9Cl);
ddd. 4-(8-Benzol[1,2,5] oxadiazol-5-yl-[1,7]napthyridin-6yl)-benzoic acid;
eee. 3-[4-(3-Chloro-phenyl)-1-ethyl-7-methyl-2-oxo-1,2,-dihdro-[1,8]naphthyridin-3-yl]-prpionamidine;
fff. 4H-[1,2,4]Triazole[5,1-b]purin-5(6H)-one, 7-cyclopentyl-2-(1 methylethyl)-4-propyl (9Cl);
ggg. Acetronitrile, (6-ethoxy-3,4-dihydro-7-methoxy-4,4-dimethyl-1 (2H)-isoquinolinylidene)[[2-(4-morpholinyl)ethyl[thio]-(9Cl);
hhh. 1-Piperidinepentanenitrile[(4aR,10bR)-9-ethoxy-1,3,4,4a,5, 1 Ob-hexahydro-8-methoxy-6(2H)-phenanthridinylidene]-, rel-(9Cl);
iii. 2H-Pyran-2-one,tetrahydro-5-phenyl-3-(phenylmethyl)-,trans-(9Cl);
jjj. 2-Pyrrolidinone, 4-[3-(cyclopentyloxy)-4-methoxyphenyl]-3-[[3-methoxy-4-(phenylmethoxy)phenyl]methyl]-(9Cl);
kkk. 4-{3-[9-(3-Cyclopentyloxy-4-methoxy-benzyl)-6,8-dimethyl-9H-purin-2-yloxy]-propyl}-propyl}-pyridine 1-oxide;
lll. Urea[2-[6,7-dihydro-9,10-dimethoxy-4-oxo-2-[(2,4,6-trimethylphenyl)imino]-2H-pyrimido[6,1-a]isoquinolin-3(4H)-yl]ethyl]-(9Cl);
mmm. 4H-Pyrimido[6,1-a]isoquinolin-4-one,2-[2,6-bis(1-methylethyl)phenoxy]-6,6-dihydro-9,10-dimethoxy-(9Cl);
nnn. 8-(3-Azido-phenyl)-6-imidazol-1-ylmeyhyl-quinoline; and
ooo. 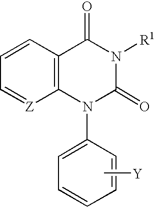
or a pharmaceutically-acceptable acid-addition salt thereof, wherein:
R₁ is hydrogen, alkyl of 1 to 3 carbon atoms, cyclopentylmethyl, cyclohexylmethyl, norbornylmethyl, [2.2.2]bicyclooctylmethyl or benzyl, the phenyl of the benzyl optionally being substituted by halogen; trifluoromethyl, nitor, carboxy or CO₂^{θ}M^{⊕} wherein M^{⊕} is a pharmaceutically aceptable cation;
Y is carboxy, carboalkoxy wherein the alkoxy has 1 to 6 carbon atoms, carbobenzyloxy, N-alkylcarboxamido wherein the alkyl has 1 to 6 carbon atoms, or CO₂^{θ}M^{θ} wherein M^{θ} is as defined above;
and Z is N or CH, provided that (i) when Z is CH, then R₁, is benzyl, Y is in the meta-position and Y may also be tetrazolyl optionally substituted by a group selected from alkyl of 1 to 3 carbon atoms and benzyl; (ii) when Z is N, Y is in the meta-or para-position of the 1-phenyl group and (iii) when R₁ is substituted benzyl, the substitution is at the meta-and/or para-positions.
